(19) United States
(12) Patent Application Publication
(10) Pub. No.: US 2009/0258823 A1
(43) Pub. Date: Oct. 15, 2009

(54) COMPOSITION AND METHODS FOR THE PREVENTION AND TREATMENT OF GASTROINTESTINAL INFECTIONS

(76) Inventors: Caroline L.S. George, Roseville, MN (US); Jeanne M. Snyder, Iowa City, IA (US); Fred S. Lamb, Coralville, IA (US)

Correspondence Address: FOLEY & LARDNER LLP, 150 EAST GILMAN STREET, P.O. BOX 1497, MADISON, WI, 53701-1497, US

(21) Appl. No.: 11/952,508

(22) Filed: Dec. 7, 2007

Related U.S. Application Data
(60) Provisional application No. 60/869,303, filed on Dec. 8, 2006.

(51) Int. Cl. A61K 38/16; A61P 1/00

(52) U.S. Cl. 514/12

(57) ABSTRACT

Methods for the prevention and treatment of gastrointestinal infections are described. Surfactant-associated protein-A (SP-A) or SP-D or an active fragment or derivative thereof is administered to mammals at risk of a gastrointestinal infection. Oral compositions of SP-A and/or SP-D are disclosed. Also disclosed is a method to prevent or treat a gastrointestinal infection in a mammal using a gene expression vector comprising the SP-A or SP-D structural gene is introduced into a mammalian cell. Also disclosed is a method to prevent or treat a gastrointestinal infection in a mammal by increasing the intestinal expression of endogenous SP-A or SP-D.

### CROSS-REFERENCE TO RELATED APPLICATIONS

This application claims priority to U.S. provisional patent application No. 60/869,303, filed Dec. 8, 2006, the entire disclosure of which is incorporated herein by reference.

### GOVERNMENT RIGHTS

This invention was made with United States Government support awarded under NIEHS/NIH P30 ES05605. The United States Government has certain rights in this invention.

### FIELD OF THE INVENTION

The invention relates generally to methods of preventing and treating gastrointestinal infections. More specifically, the invention relates to the prevention and treatment of gastrointestinal infections and/or pathogen translocation across the intestine wall in at-risk individuals using surfactant associated proteins such as surfactant-associated protein-A and surfactant-associated protein D.

### BACKGROUND

The following description is provided to assist the understanding of the reader. None of the information provided or references cited is admitted to be prior art to the present invention.

The gastrointestinal tract of newborns is sterile at birth and rapidly becomes colonized with bacteria. Many of these bacteria are potentially dangerous pathogens which, if allowed to gain access to the bloodstream, could result in systemic disease. In a newborn, necrotizing enterocolitis (NEC) is a syndrome associated with prematurity, altered intestinal microbial colonization, ischemia, and breakdown of the intestinal mucosal barrier resulting in translocation of pathogens. NEC is characterized by coagulation or ischemic necrosis on pathologic examination. NEC causes significant morbidity and mortality in the pediatric population, especially in at risk infants and newborns such as those born prematurely (i.e., less than 32 weeks gestation), those with medical illnesses and those with congenital disease requiring surgery.

Immunocompromised or certain other older individuals may also be at risk of sepsis from gastrointestinal infections. For example, typhlitis (nectrotizing colitis) affects immunocompromised patients, such as those undergoing chemotherapy, patients with AIDS, kidney transplant patients, or the elderly. The condition is usually caused by gram negative commensal enteric bacteria, but can also include gram positive bacteria and yeast.

Surfactant-associated protein-A (SP-A) and surfactant-associated protein-D (SP-D) are collecting, a family of proteins characterized by collagenous- and carbohydrate-binding domains. SP-A and SP-D have unique, highly conserved sequences. U. Kishore et al. Molecular Immunology, 43: 1293-1315 (2006). SP-A is an approximately 248 amino acid polypeptide comprising four major domains: a cysteine-containing N-terminal domain; a collagen-like domain; a neck region; and a C-terminal C-type lectin carbohydrate recognition domain. SP-A monomers assemble into a complex oligomeric tertiary structure, with extensive post-translational modifications, including sulfation, acetylation, hydroxylation of prolines, and addition of complex polysaccharides. In humans, SP-A is encoded by two genes, SP-A1 and SP-A2, Native alveolar SP-A is believed to assemble as a heterotrimer comprised of two subunits of SP-A1 and one subunit of SP-A2. Six heterotrimers then bind together to form a “flower bouquet” structure. U. Kishore. et al., Molecular Immunology, 43: 1293-1315 (2006).

SP-A is produced at various sites throughout the body. SP-A was first discovered in the lung and was identified as the most abundant protein in pulmonary surfactant, which is a lipoprotein that covers the alveolar surface. SP-A has been implicated in performing several functions in the lung. As a component of surfactant, SP-A helps to reduce surface tension under reduced phospholipid concentrations and an absence of SP-A is associated with less tubular myelin figures.” T R Korfliagen et al., Proc Natl Acad Sci USA. 1996; 93(18): 9594-9599. Subsequent studies have shown SP-A to be present in the gastrointestinal tract, peritoneal cavity, middle ear, and other tissues. B. A. W. M. van Rozendaal et al. Ped Path Mol Med 20: 319-339 (2001); Madsen et al. 2003. Am J Respir Cell Mol Biol 29:591-597. For a general review of SP-A, see K. R. Khubchandani & J. M. Snyder, Faseb J 15: 59-69 (2001). Likewise, SP-D has also been detected in murine tissues such as the lung, esophagus, trachea, salivary gland, lacrimal gland, ovary and uterus. SP-D has also been detected in small amounts in human lung, gastrointestinal, renal and urinary tracts. See, e.g., Crouch et al. American Journal of Respiratory Cell and Molecular Biology. 35: 84-94, 2006; Madsen et al. 2000. J Immunol 164:5866-5870.

### SUMMARY

It has unexpectedly been discovered that collectins such as SP-A and SP-D may be used to treat or prevent gastrointestinal infections in mammals. In particular, collectins may be used to treat or prevent gastrointestinal infections or pathogen translocation across the intestinal wall in at-risk individuals, including, but not limited to, newborn infants, immunocompromised individuals, cancer patients, and critically ill patients in an ICU setting. In accordance with one aspect, the invention provides a method of administering a therapeutically effective amount of one or more isolated surfactant-associated proteins to a mammal having or at risk for having a gastrointestinal infection. In some embodiments, these collectins will be surfactant-associated proteins. Surfactant-associated proteins suitable for use in methods of the invention include SP-A, SP-D, an active fragment of SP-A, an active fragment of SP-D, an active derivative of SP-A, and an active derivative of SP-D, each active fragment or derivative having at least 90%, at least 95%, at least 98%, or at least 99% amino acid sequence identity to an SP-A or SP-D protein. The one or more surfactant-associated proteins can be administered prior to or after the onset of infection. For example, a mammal at risk of having a gastrointestinal infection includes a newborn infant where the GI tract has not yet been colonized with bacteria.

In some embodiments of methods of treating or preventing a gastrointestinal infection, the infection is caused by a viral pathogen. In other embodiments, the infection is caused by a bacterial pathogen such as Klebsiella oxytoca, Klebsiella pneumonia, Enterobacter sp., Clostridium, Pseudomonas putida, E. coli, Group B streptococci, Listeria, Staphylococcus aureus, Salmonella, and Bacillus sp. In some embodiments, the methods are directed to the prevention or treatment of necrotizing enterocolitis (NEC). In other embodiments, the methods are directed to the prevention or treatment of typhlitis.

In some embodiments of methods described herein, the mammal is a human, rat, cat, dog, cow, pig, mouse, equine, or primate. In certain embodiments, the mammal is a human infant.

In another aspect of the invention, compositions are provided comprising one or more surfactant-associated proteins, wherein the surfactant-associated protein is selected from the group consisting of surfactant-associated protein-A (SP-A), surfactant associated protein-D (SP-D), an active fragment of SP-A, an active fragment of SP-D, an active derivative of SP-A, and an active derivative of SP-D, each active fragment or derivative having at least 90%, at least 95%, at least 98%, or at least 99% amino acid sequence identity to an SP-A or SP-D protein, and wherein the one or more surfactant-associated proteins are present in a therapeutically effective amount for the prevention or treatment of gastrointestinal infections in mammals. In some embodiments, the composition is administered within 7 days of birth. Such compositions may be in powdered form for storage and transport and may be administered as a solid or in a fluid suitable for oral administration, e.g., via infant formula or milk. The infant formula may be in powder or liquid form. In some embodiments, the therapeutically effective amount of one or more surfactant-associated proteins, such as SP-A and/or SP-D, is from about 0.01 mg/kg to about 2 mg/kg.

In some embodiments, the surfactant-associated protein is human, rat, or bovine SP-A or SP-D. The human SP-A can be SP-A1 or SP-A2. In some embodiments, the surfactant associated protein is SP-A1, having an amino acid sequence selected from SEQ ID NO: 3, an active fragment thereof or an active derivative thereof, wherein the active fragment or derivative has at least 90%, at least 95%, at least 98%, or at least 99% amino acid sequence identity to SEQ ID NO: 3. In other embodiments, the surfactant associated protein is SP-A2, having an amino acid sequence selected from SEQ ID NO: 4, an active fragment thereof or an active derivative thereof, wherein the active fragment or derivative has at least 90%, at least 95%, at least 98%, or at least 99% amino acid sequence identity to SEQ ID NO: 4. In yet other embodiments, the SP-A is a heterotrimer of two molecules of SP-A1 and one molecule of SP-A2. In other embodiments, the surfactant associated protein is SP-D, having an amino acid sequence selected from SEQ ID NO: 6, an active fragment thereof or an active derivative thereof, wherein the active fragment or derivative has at least 90%, at least 95%, at least 98%, or at least 99% amino acid sequence identity to SEQ ID NO: 6.

### DESCRIPTION OF THE FIGURES

FIG. 1 is a chart showing SP-A null and heterozygous mice pedigrees.

FIG. 2 is a chart showing small bowel histology for wild type and SP-A null mice exposed to control and corn dust bedding.

FIG. 3 is a Kaplan-Meier survival analysis graph of mouse wild-type, heterozygous, and SP-A null mice exposed to control and corn dust bedding.

FIG. 4 is a Kaplan-Meier Survival Analysis graph of SP-A null mice administered exogenous human SP-A protein after birth.

FIG. 5 is a graph showing the level of SP-D gene expression in lung, lactating, and non-lactating mammary gland tissues.

FIG. 6 is a western blot of SP-D proteins in mammary tissues and milk samples.

### DETAILED DESCRIPTION

In the description that follows, a number of terms are utilized extensively. Definitions are herein provided to facilitate understanding of the invention. The terms defined below are more fully defined by reference to the specification as a whole.

Units, prefixes, and symbols may be denoted in their accepted SI form. Unless otherwise indicated, nucleic acids are written left to right in 5′ to 3′ orientation; amino acid sequences are written left to right in amino to carboxy orientation. Amino acids may be referred to herein by either their commonly known three letter symbols or by the one-letter symbols recommended by the IUPAC-IUBMB Nomenclature Commission. Nucleotidcs, likewise, may be referred to by their commonly accepted single-letter codes.

As used herein, an “active fragment” of a polypeptide is one having an in vivo or in vitro biological activity which is characteristic of naturally occurring collectin polypeptides from which the fragment is derived. Fragments may arise from post-transcriptional processing, from translation of alternatively spliced RNAs, from the selective expression of a portion of the entire polypeptide, or the addition of a tag, linker, or other sequence to the N- or C-terminus of the protein. Fragments include those expressed in native or endogenous cells as well as those made in expression systems. Fragments of a nucleotide sequence may encode protein fragments that retain the biological activity of the native protein.

As used herein, the terms “administer,” “administering,” or “administration” refer to any route of introducing or delivering to a subject a compound to perform its intended function, Administration can be carried out by any suitable route, including orally, intranasally, parenterally (intravenously, intramuscularly, intraperitoneally, or subcutaneously), rectally, or topically.

The terms “derivative” or “variant” of a polypeptide refer to a polypeptide which differs from a naturally occurring polypeptide in amino acid sequence or in ways that do not involve amino acid sequence modifications, or both. Non-sequence modifications include, but are not limited to, changes in acetylation, methylation, phosphorylation, carboxylation, or glycosylation. Derivatives may also include sequences that differ from the wild-type sequence by one or more conservative amino acid substitutions or by one or more non-conservative amino acid substitutions, deletions, or insertions which do not substantially diminish or at least do not completely destroy the biological activity of the polypeptide. Conservatively modified variants typically provide similar biological activity as the unmodified polypeptide sequence from which the conservatively modified variant was derived. Conservative substitutions typically include the substitution of one amino acid for another with similar characteristics such as hydrophobic, polar, acidic or basic side chains. Conservative substitution tables providing functionally similar amino acids are well known in the art. For example, the following six groups each contain amino acids that are conservative substitutions for one another: Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic: Arginine (R), Lysine (K), Histidine (H); Acidic: Aspartic acid (D), Glutamic acid (E); Polar: Serine (S), Threonine (T), Asparagine (N), Glutamine (Q). Likewise, for nucleotide sequences, conservative variants include those sequences that, because of the degeneracy of the genetic code, encode the amino acid sequence of one of the polypeptides of the invention. Nucleotide variants may be natural or synthetic mutants generated, for example, by site-directed mutagenesis. An active derivative or variant of a polypeptide herein retains measurable biological activity utilizing one or more of the assays and/or other tests and procedures described herein. In some embodiments, the active derivative retains at least 0.1%, at least 1%, at least 10%, at least 20%, at least 30%, at least 40%, at least 50%, at least 60%, at least 70%, at least 80%, at least 90%, at least 95%, at least 98% or at least 99% of the activity of the native polypeptide. Bacterial binding, bacterial aggregation, bacterial permeability and phagosytosis assays may be used to determine the activity of SP-A and SP-D, and active fragments and derivatives thereof. Examples of such assays are described in W. Watford et al. 2002 Am J Physiol Lung Cell Mol Physiol, 283: L101-L1022; Wu et al. 2003. J Clin Invest 111:1589-1602; Prostate 65: 241-51 (2005); Mol Hum Reprod 10: 861-70 (2004); Am J Physiol Lung Cell Mol Physiol 287: L296-306 (2004).

As used herein, the terms “effective amount,” “pharmaceutically effective amount” and “therapeutically effective amount” of a composition refer to a quantity sufficient to achieve a desired therapeutic and/or prophylactic effect. In the context of treating a disease or condition, a “therapeutically effective amount” is an amount which results in the prevention of, or a decrease in, the symptoms associated with a disease or condition that is being treated. The amount of a composition of the invention administered to the subject will depend on the type and severity of the disease and on the characteristics of the individual, such as general health, age, sex, body weight and tolerance to drugs. It will also depend on the degree, severity and type of disease. Those of skill in the art will be able to determine appropriate dosages depending on these and other factors. The compositions of the present invention can also be administered in combination with one or more additional therapeutic compounds. In some embodiments, a collectin polypeptide, e.g., SP-A or SP-D, is administered in an amount which alleviates, in whole or in part, symptoms associated with a gastrointestinal infection, or halts of further progression or worsening of those symptoms, or prevents or provides prophylaxis for the gastrointestinal infection in a subject at risk for developing a gastrointestinal infection. In one embodiment, an effective amount of a collectin polypeptide, e.g., SP-A or SP-D, is administered in an amount which protects a subject from the symptoms of necrotizing enterocolitis (NEC), wherein subjects administered the effective amount of collectin polypeptide show a reduce occurrence of NEC compared to subjects not administered the effective amount of collectin polypeptide.

As used herein, the term “endogenous” refers to a gene, protein, or other substance derived or originating from the organism or cell.

As used herein, the term “exogenous” refers to a protein or other substance derived or originating external to the organism or cell.

As used herein, the term “expression” refers to the process by which a polypeptide is produced from a structural gene. Overall, the process involves transcription of a gene into RNA and the translation of such RNA into polypeptide(s). However, as used herein, expression may also cover translation of synthetic RNA into polypeptide.

As used herein, the terms “isolated” or substantially purified” refers to material, such as a nucleic acid or a protein, which is substantially or essentially free from components which normally accompany or interact with it as found in its natural environment. The isolated material optionally comprises material not found with the material in its natural environment. Alternatively, if the material is in its natural environment, the material may be isolated if it has been synthetically altered or synthetically produced by deliberate human intervention and/or placed at a different location within the cell. Moreover, a naturally-occurring nucleic acid becomes isolated if it is introduced to a different locus of the genome. In some cases, a substantially pure protein will be evidenced by a single band following sodium dodecyl sulfate polyacrylamide gel electrophoresis (SDS PAGE). The term “substantially pure” is further meant to describe a molecule which is homogeneous by one or more purity or homogeneity characteristics used by those of skill in the art.

As used herein, the term “operably linked” refers to a functional linkage between two sequences. For example, when a promoter and a structural gene are joined, the promoter sequence initiates and mediates transcription of the structural gene. Generally, operably linked means that the nucleic acid sequences being linked are contiguous and, where necessary to join two protein coding regions, contiguous and in the same reading frame.

As used herein, the terms “preventing” or “prevention” refer to a reduction in risk of acquiring a disease or disorder (i.e., causing at least one of the clinical symptoms of the disease not to develop in a patient that may be exposed to or predisposed to the disease but does not yet experience or display symptoms of the disease). In the context of the present invention, prevention includes interfering with the mechanism by which pathogens cause gastrointestinal infections. For example, one mechanism of action for the compositions of the present invention may include effective clearing of pathogens from the intestinal tract prior to infection.

As used herein, the terms “polynucleotide” or “nucleic acid” refer to a deoxyribopolynucleotide, ribopolynucleotide, or chimeras or analogues thereof in single-strand or double-strand form that have the essential nature of a natural deoxy- or ribonucleotide in that they hybridize, under stringent hybridization conditions, to substantially the same nucleotide sequence as naturally occurring nucleotides and/or allow translation into the same amino acid(s) as the naturally occurring nucleotide(s). Unless otherwise indicated, the term includes reference to the specified sequence as well as the complementary sequence thereof. DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically, or metabolically modified forms of polynucleotides, as well as the chemical forms of DNA and RNA characteristic of viruses and cells, including among other things, simple and complex cells.

As used herein, the terms “polypeptide,” “peptide,” and “protein” are used interchangeably to refer to a polymer of amino acid residues. The terms apply to amino acid polymers in which one or more amino acid residues is an artificial chemical analog of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. The terms polypeptide, peptide, and protein are also inclusive of modifications including, but not limited to, glycosylation, lipid attachment, sulfation, carboxylation, hydroxylation, ADP-ribosylation, and addition of other complex polysaccharides.

As used herein, the term “promoter” refers to a DNA sequence which directs the transcription of a structural gene to produce a messenger RNA (mRNA). Typically, a promoter is located in the 5′ region of a gene, proximal to the start codon of a structural gene. If a promoter is an inducible promoter, then the rate of transcription increases in response to an inducing agent. In contrast, if a promoter is a constitutive promoter, the rate of transcription is not regulated by an inducing agent.

As used herein, the term “recombinant” refers to a cell or vector that has been modified by the introduction of a heterologous nucleic acid or that the cell is derived from a cell so modified. Thus, for example, recombinant cells express genes that are not found in identical form within the native (non-recombinant) form of the cell or express native genes that are otherwise abnormally expressed, under-expressed, or not expressed at all as a result of human intervention. A protein expressed from a recombinant vector is a recombinant protein.

As used herein, the term “subject” means that preferably the subject is a mammal, preferably a human, but can also be an animal such as a domestic animal (e.g., dogs, cats and the like), farm animal (e.g., cows, sheep, pigs, horses and the like) or laboratory animal (e.g., monkey, rats, mice, rabbits, guinea pigs and the like).

As used herein, the terms “treating,” “treatment” and “alleviation” refer to both therapeutic treatment and prophylactic or preventative measures, wherein the object is to prevent or slow down (lessen) the targeted pathologic condition or disorder. A subject is successfully “treated” for a disorder if the subject shows observable and/or measurable reduction in or absence of one or more signs and symptoms of a particular disease or condition.

The terms “transfected,” “transformed” and “transduced,” as used herein, refer to a process by which exogenous nucleic acid is transferred or introduced into the host cell. A “transfected,” “transformed” or “transduced” cell is one which has been transfected, transformed or transduced with exogenous nucleic acid. The cell includes the primary subject cell and its progeny.

The terms “sequence identity” and “percentage identity” refer to the residues in two sequences which are the same when aligned for maximum correspondence over a specified comparison window. When percentage of sequence identity is used in reference to proteins, it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Thus, where sequences differ in conservative substitutions, the percent sequence identity may be adjusted upwards to correct for the conservative nature of the substitution. Sequences which differ by such conservative substitutions are said to have “sequence similarity” or “similarity.” Methods of alignment of sequences for comparison are well-known in the art. For instance, alignment may be made using publicly available computer software such as BLAST, BLAST-2, ALIGN or Megalign (DNASTAR) software; or GAP, BESTFIT, BLAST, FASTA, and TFASTA in the Wisconsin Genetics Software Package, Genetics Computer Group (GCG), Madison, Wis. Those skilled in the art can determine appropriate parameters for measuring alignment, including any algorithms needed to achieve maximal alignment over the full length of the sequences being compared.

The present invention provides methods and compositions for using collectins such as SP-A and SP-D to prevent or treat gastrointestinal infections. In an exemplary embodiment, immunocompromised or at-risk human infants are administered SP-A and/or SP-D to prevent or treat gastrointestinal infections. Alternatively, immunocompromised individuals, such as chemotherapy patients, transplant patients taking immunosuppressant drugs, and patients in an ICU setting may be administered SP-A and/or SP-D as a means to treat or prevent gastrointestinal infections. In another embodiment, other mammals at risk for gastrointestinal infections, including, but not limited to livestock and pets, may benefit from the administration of collectins to prevent or treat gastrointestinal infections.

In one embodiment, newborn infants are administered SP-A and/or SP-D as a supplement, e.g., to their milk or infant formula. The surfactant-associated proteins may be incorporated into infant formula or milk according to well-known procedures for supplementing infant formula, e.g., as described in U.S. Patent Publication Nos. 2006/0247153, 2006/0233915, 2006/0233752, 2006/0233762, 2006/0275909, 2006/0210692, and 2006/0210697, the entire contents of each of which are incorporated by reference herein. Immunocompromised newborn babies, including all premature infants, are likely to benefit from supplemental SP-A and/or SP-D because no bacteria have yet colonized their gastrointestinal tract. This population is at risk for developing a systemic infection by gastrointestinal bacteria or viruses. Accordingly, in some embodiments, SP-A and/or SP-D would be administered to these patients immediately or shortly after birth (e.g., within 1, 2, 6, 12, or 24 hours, or within 2, 3, 4, 5, 6, or 7 days of birth), before their GI tract becomes colonized with bacteria. In some embodiments, the treatment could continue indefinitely or as long as the patients are at risk for a gastrointestinal infection. Alternatively, SP-A and/or SP-D can be administered after the onset of infection to prevent progression of the disease or lessen its symptoms. If not provided in milk or infant formula, the SP-A and/or SP-D can be administered in the form of an oral suspension, oral emulsion, powder, capsule, or tablet.

In one aspect, a therapeutically effective dose of SP-A and/or SP-D is administered to a patient. A therapeutically effective dose can vary depending upon the route of administration and dosage form. The exact dose is chosen by a physician in view of the condition of a patient to be treated. Doses and administration are adjusted to provide a sufficient level of the active portion of SP-A and/or SP-D, or to maintain a desired effect. Specific dosages can be adjusted depending on conditions of disease, the age, body weight, general health conditions, sex, and diet of the subject, dose intervals, administration routes, excretion rate, and combinations of drugs. In some embodiments, an effective amount of SP-A or SP-D will range from about 0.01 mg/kg to about 2 mg/kg of body weight. In other embodiments, an effective amount of SP-A will range from about 0.1 to about 2 mg/kg, from about 0.1 to 1 mg/kg of body weight, or from any of about 0.05, 0.1, 0.2, 0.3, or 0.4 mg/kg of body weight to any of about 0.5, 0.6, 0.7, 0.8, 0.9 or 1.0 mg/kg of body weight. In other embodiments, the effective amount can be less than or equal to 2.0, 1.5, 1.0, 0.5, or 0.25 mg/kg of body weight.

In an exemplary embodiment, the SP-A protein of the present invention is produced in substantially purified form using conventional molecular biology or biochemical techniques (Kuzmenko, W. H. et al., J Clin Invest 111:1589-1602 (2003). However, alternative embodiments may use SP-A isolated from other sources, such as from the pulmonary surfactant of animals. Typically however, the protein of the present invention is expressed by recombinant E. coli carrying a nucleic acid construct for expression of SP-A. The making of an expression vector involves inserting the SP-A structural gene into an expression system to which the nucleic acid molecule is heterologous (i.e., not normally present). The heterologous nucleotide may be selected from the group consisting of SEQ ID NO: 1 (Table 1), SEQ ID NO:2 (Table 2). Sequences of SP-D suitable for use in methods described herein include SEQ ID NO: 5 (Table 3) and may be found at Rust, K. et al., Arch. Biochem. Biophys. 290: 116-126 (1991); Lu, J. et al. Biochem. J. 284: 785-802 (1992); and Crouch, E. et al. J. Biol. Chem. 268: 2976-2983 (1993). The heterologous nucleic acid molecule is inserted into the expression system which includes the necessary elements for the transcription and translation of the inserted protein-coding sequences.

TABLE 1
Nucleotide Sequence of Human SP-A1: [SEQ ID NO: 1]

ATGTGGCTGTGCCCTCTGGCCCTCAACCTCATCTTGATGGCAGCCTCTGGTGCTGTGTGCGAAGTGAAGG
ACGTTTGTGTTGGAAGCCCTGGTATCCCCGGCACTCCTGGATCCCACGGCCTGCCAGGCAGGGACGGGAG
AGATGGTCTCAAAGGAGACCCTGGCCCTCCAGGCCCCATGGGTCCACCTGGAGAAATGCCATGTCCTCCT
GGAAATGATGGGCTGCCTGGAGCCCCTGGTATCCCTGGAGAGTGTGGAGAGAAGGGGGAGCCTGGCGAGA
GGGGCCCTCCAGGGCTTCCAGCTCATCTAGATGAGGAGCTCCAAGCCACACTCCACGACTTTAGACATCA
AATCCTGCAGACAAGGGGAGCCCTCAGTCTGCAGGGCTCCATAATGACAGTAGGAGAGAAGGTCTTCTCC
AGCAATGGGCAGTCCATCACTTTTGATGCCATTCAGGAGGCATGTGCCAGAGCAGGCGGCCGCATTGCTG
TCCCAAGGAATCCAGAGGAAAATGAGGCCATTGCAAGCTTCGTGAAGAAGTACAACACATATGCCTATGT
AGGCCTGACTGAGGGTCCCAGCCCTGGAGACTTCCGCTACTCAGACGGGACCCCTGTAAACTACACCAAC
TGGTACCGAGGGGAGCCCGCAGGTCGGGGAAAAGAGCAGTGTGTGGAGATGTACACAGATGGGCAGTGGA
ATGACAGGAACTGCCTGTACTCCCGACTGACCATCTAG

TABLE 2
Nucleotide Sequence of Human SP-A2: [SEQ ID NO: 2]

ATGTGGCTGTGCCCTCTGGCCCTCACCCTCATCTTGATGGCAGCCTCTGGTGCTGCGTGCGAAGTGAAGG
ACGTTTGTGTTGGAAGCCCTGGTATCCCCGGCACTCCTGGATCCCACGGCCTGCCAGGCAGGGACGGGAG
AGATGGTGTCAAAGGAGACCCTGGCCCTCCAGGCCCCATGGGTCCGCCTGGAGAAACACCATGTCCTCCT
GGGAATAATGGGCTGCCTGGAGCCCCTGGTGTCCCTGGAGAGCGTGGAGAGAAGGGGGAGGCTGGCGAGA
GAGGCCCTCCAGGGCTTCCAGCTCATCTAGATGAGGAGCTCCAAGCCACACTCCACGACTTCAGACATCA
AATCCTGCAGACAAGGGGAGCCCTCAGTCTGCAGGGCTCCATAATGACAGTAGGAGAGAAGGTCTTCTCC
AGCAATGGGCAGTCCATCACTTTTGATGCCATTCAGGAGGCATGTGCCAGAGCAGGCGGCCGCATTGCTG
TCCCAAGGAATCCAGAGGAAAATGAGGCCATTGCAAGCTTCGTGAAGAAGTACAACACATATGCCTATGT
AGGCCTGACTGAGGGTCCCAGCCCTGGAGACTTCCGCTACTCAGATGGGACCCCTGTAAACTACACCAAC
TGGTACCGAGGGGAGCCTGCAGGTCGGGGAAAAGAGCAGTGTGTGGAGATGTACACAGATGGGCAGTGGA
ATGACAGGAACTGCCTGTACTCCCGACTGACCATCTGTGAGTTCTGA

TABLE 3
Nucleotide Sequence of Human SP-D: [SEQ ID NO: 5]

ATGCTGCTCTTCCTCCTCTCTGCACTGGTCCTGCTCACACAGCCCCTGGGCTACCTGGAAGCAGAAATGA
AGACCTACTCCCACAGAACAATGCCCAGTGCTTGCACCCTGGTCATGTGTAGCTCAGTGGAGAGTGGCCT
GCCTGGTCGCGATGGACGGGATGGGAGAGAGGGCCCTCGGGGCGAGAAGGGGGACCCAGGTTTGCCAGGA
GCTGCAGGGCAAGCAGGGATGCCTGGACAAGCTCGCCCAGTTGGGCCCAAAGGGGACAATGGCTCTGTTG
GAGAACCTGGACCAAAGGGAGACACTGGGCCAAGTGGACCTCCAGGACCTCCCGGTGTGCCTGGTCCAGC
TGGAAGAGAAGGTCCCCTGGGGAAGCAGGGGAACATAGGACCTCAGGGCAAGCCAGGCCCAAAAGGAGAA
GCTGGGCCCAAAGGAGAAGTAGGTGCCCCAGGCATGCAGGGCTCGGCAGGGGCAAGAGGCCTCGCAGGCC
CTAAGGGAGAGCGAGGTGTCCCTGGTGAGCGTGGAGTCCCTGGAAACACAGGGGCAGCAGGGTCTGCTGG
AGCCATGGGTCCCCAGGGAAGTCCAGGTGCCAGGGGACCCCCGGGATTGAAGGGGGACAAAGGCATTCCT
GGAGACAAAGGAGCAAAGGGAGAAAGTGGGCTTCCAGATGTTGCTTCTCTGAGGCAGCAGGTTGAGGCCT
TACAGGGACAAGTACAGCACCTCCAGGCTGCTTTCTCTCAGTATAAGAAAGTTGAGCTCTTCCCAAATGG
CCAAAGTGTCGGGGAGAAGATTTTCAAGACAGCAGGCTTTGTAAAACCATTTACGGAGGCACAGCTGCTG
TGCACACAGGCTGGTGGACAGTTGGCCTCTCCACGCTCTGCCGCTGAGAATGCCGCCTTGCAACAGCTGG
TCGTAGCTAAGAACGAGGCTGCTTTCCTGAGCATGACTGATTCCAAGACAGAGGGCAAGTTCACCTACCC
CACAGGAGAGTCCCTGGTCTATTCCAACTGGGCCCCAGGGGAGCCCAACGATGATGGCGGGTCAGAGGAC
TGTGTGGAGATCTTCACCAATGGCAAGTGGAATGACAGGGCTTGTGGAGAAAAGCGTCTTGTGGTCTGCG
AGTTCTGA

The heterologous nucleotide may also encode other suitable collecting, including those from other species. In one embodiment, a nucleotide encoding bovine SP-A or SP-D is used. The sequence encoding bovine SP-A is shown in Table 4 and the sequence encoding bovine SP-D is shown in Table 5.

TABLE 4
Nucleotide Sequence of Bovine SP-A: [SEQ ID NO: 11]

ATGCTGCTGTGCTCTTTGACCCTTACCCTCCTCTGGATGGTGGCTTCTGGCCTCGAGTGCGATGTCAAGG
AAGTTTGTCTTGGAAGCCCTGGCATTCCTGGCACTCCTGGATCCCATGGCCTGCCAGGAAGAGATGGGAG
AGATGGTATCAAAGGAGACCCTGGGCCTCCAGGCCCCATGGGCCCCCCTGGAGGAATGCCAGGCCTCCCT
GGGCGTGATGGGATGACTGGAGCCCCTGGCCTCCCTGGAGAGCGTGGAGAAAAGGGAGAGCCTGGCGAGA
GAGGTCCTCCAGGGTTTCCAGCATATCTAGATGAAGAGCTCCAGGGCACACTCCATGAGATCAGACATCA
AGTCCTGCAGTCACAGGGCGTCCTCCGTTTGCAGGGGTCCGTGCTGGCGGTGGGAGAGAAGGTCTTCTCT
ACCAATGGGCAGTCAGTCAATTTTGATGCCATTAAAGAGTTATGTGCCAGAGTAGGTGGACATATTGCTG
CCCCGAGGAGTCCAGAGGAGAATGAAGCCATTGTGAGCATCGTGAAGAAGTACAACACTTATGCTTACCT
GGGCCTGGTCGAAGGCCCCACCGCTGGAGACTTCTATTACCTGGATGGAGCCCCTGTGAATTATACCAAT
TGGTACCCAGGGGAGCCCAGGGGCCGGGGTAAAGAGAAGTGTGTAGAAATATACACAGATGGTCAGTGGA
ATGACAAGAACTGCCTGCAGTACCGACTGGCCATCTGTGAGTTCTGA

TABLE 5
Nucleotide Sequence of Bovine SP-D: [SEQ ID NO: 12]

ATGTGGCTGTGCCCTCTGGCCCTCAACCTCATCTTGATGGCAGCCTCTGGTGCTGTGTGCGAAGTGAAGG
ACGTTTGTGTTGGAAGCCCTGGTATCCCCGGCACTCCTGGATCCCACGGCCTGCCAGGCAGGGACGGGAG
AGATGGTCTCAAAGGAGACCCTGGCCCTCCAGGCCCCATGGGTCCACCTGGAGAAATGCCATGTCCTCCT
GGAAATGATGGGCTGCCTGGAGCCCCTGGTATCCCTGGAGAGTGTGGAGAGAAGGGGGAGCCTGGCGAGA
GGGGCCCTCCAGGGCTTCCAGCTCATCTAGATGAGGAGCTCCAAGCCACACTCCACGACTTTAGACATCA
AATCCTGCAGACAAGGGGAGCCCTCAGTCTGCAGGGCTCCATAATGACAGTAGGAGAGAAGGTCTTCTCC
AGCAATGGGCAGTCCATCACTTTTGATGCCATTCAGGAGGCATGTGCCAGAGCAGGCGGCCGCATTGCTG
TCCCAAGGAATCCAGAGGAAAATGAGGCCATTGCAAGCTTCGTGAAGAAGTACAACACATATGCCTATGT
AGGCCTGACTGAGGGTCCCAGCCCTGGAGACTTCCGCTACTCAGACGGGACCCCTGTAAACTACACCAAC
TGGTACCGAGGGGAGCCCGCAGGTCGGGGAAAAGAGCAGTGTGTGGAGATGTACACAGATGGGCAGTGGA
ATGACAGGAACTGCCTGTACTCCCGACTGACCATCTAG

The present invention utilizes polypeptides and polynucleotides that encode collectins such as SP-A or SP-D. It is not intended that the invention be limited to the sequences shown herein, as alternative embodiments may use other collectin polypeptide or polynucleotide sequences. In addition, there are many alleles of the various human collecting, which vary in amino acid sequence. Any suitable collectin polypeptide and/or polynucleotide may be used in accordance with the present invention, including modified or derivative human polypeptides or polynucleotides, or collectin polynucleotides and/or polypeptides isolated from other species. The human SP-A1 and SP-A2 polynucleotides and proteins have been fully described in the art and may be made using any of the methods known in the art. The coding sequences of the human SP-A1 and SP-A2 structural genes are shown herein as SEQ ID NOS:1 and 2, respectively. The amino acid sequences of human SP-A1 and SP-A2 are shown herein as SEQ ID NOS: 3 and 4 (Tables 6 and 7, respectively). The amino acid sequence of SP-D is shown herein as SEQ ID NO: 6 (Table 8).

TABLE 6
Amino Acid Sequence of Human SP-A1: [SEQ ID NO: 3]

MWLCPLALNLILMAASGAVCEVKDVCVGSPGIPGTPGSHGLPGRDGRDGLKGDPGPPGPMGPPGEMPCPPGN
DGLPGAPGIPGECGEKGEPGERGPPGLPAHLDEELQATLHDFRHQILQTRGALSLQGSIMTVGEKVFSSNGQ
SITFDAIQEACARAGGRIAVPRNPEENEAIASFVKKYNTYAYVGLTEGPSPGDFRYSDGTPVNYTNWYRGEP
AGRGKEQCVEMYTDGQWNDRNCLYSRLTICEF

TABLE 7
Amino Acid Sequence of Human SP-A2: [SEQ ID NO: 4]

MWLCPLALNLILMAASGAACEVKDVCVGSPGIPGTPGSHGLPGRDGRDGVKGDPGPPGPMGPPGETPCPPGN
NGLPGAPGVPGERGEKGEAGERGPPGLPAHLDEELQATLHDFRHQILQTRGALSLQGSIMTVGEKVFSSNGQ
SITFDAIQEACARAGGRIAVPRNPEENEAIASFVKKYNTYAYVGLTEGPSPGDFRYSDGTPVNYTNWYRGEP
AGRGKEQCVEMYTDGQWNDRNCLYSRLTICDF

TABLE 8
Amino Acid Sequence of Human SP-D: [SEQ ID NO: 6]

MLLFLLSALVLLTQPLGYLEAEMKTYSHRTMPSACTLVMCSSVESGLPGRDGRDGREGPRGEKGDPGLPGAA
GQAGMPGQAGPVGPKGDNGSVGEPGPKGDTGPSGPPGPPGVPGPAGREGALGKQGNIGPQGKPGPKGEAGPK
GEVGAPGMQGSAGARGLAGPKGERGVPGERGVPGNTGAAGSAGAMGPQGSPGARGPPGLKGDKGIPGDKGAK
GESGLPDVASLRQQVEALQGQVQHLQAAFSQYKKVELFPNGQSVGEKIFKTAGFVKPFTEAQLLCTQAGGQL
ASPRSAAENAALQQLVVAKNEAAFLSMTDSKTEGKFTYPTGESLVYSNWAPGEPNDDGGSEDCVEIFTNGKW
NDRACGEKRLVVCEF

Active fragments, derivatives, or variants of the polypeptides of the present invention may be recognized by, for example, the deletion or addition of amino acids that have minimal influence on the properties, secondary structure, and biological activity of the polypeptide. For example, a polypeptide may be joined to a signal (or leader) sequence at the N-terminal end of the protein which co-translationally or post-translationally directs subcellular or extracellular localization of the protein. The polypeptide may also be conjugated to a linker or other sequence at its N- or C-terminus for ease of synthesis, purification, or identification of the polypeptide.

The proteins and DNA sequences encoding the proteins may be produced using standard methods. Purification can also be accomplished using standard procedures such as isolating proteins using chromatography, using antibodies directed against the polypeptides, or by producing the polypeptides in a form in which they are fused to a moiety (or tag) that aids in purification and which can then be cleaved.

The SP-A or SP-D polynucleotide of the present invention may be inserted into any of the many commercially available expression vectors using reagents and techniques that are well known in the art. In preparing the recombinant expression constructs, the various polynucleotides of the present invention may be inserted or substituted into a bacterial plasmid-vector. Any convenient plasmid may be employed, which will be characterized by having a bacterial replication system, a marker which allows for selection in a bacterium and generally one or more unique, conveniently located cloning sites. Numerous plasmids, also referred to as vectors, are available for transformation. Suitable vectors include, but are not limited to, the following: viral vectors, such as lambda vector system gt11, Charon 4, and plasmid vectors such as pBR322, pBR325, pACYC177, pACYC1084, pUC8, pUC9, pUC18, pUC19, pLG339, pR290, pKC37, pKC101, SV 40, pBluescript II SK +/−, or KS +/−(Stratagene, La Jolla, Calif.), and any derivatives thereof. Also suitable are yeast expression vectors, which may be highly useful for cloning and expression. Exemplary yeast plasmids include, without limitation, pPICZ, and pFLD. (Invitrogen, Carlsbad, Calif.). The selection of a vector will depend on the preferred transformation technique and target host cells.

The nucleic acid molecule encoding SP-A or SP-D is inserted into a vector in the 5′ to 3′ direction, such that the open reading frame is properly oriented for the expression of the encoded protein under the control of a promoter of choice. In this way, the SP-A or SP-D structural gene is said to be “operably linked” to the promoter. Single or multiple nucleic acids may be inserted into an appropriate vector in this way, each under the control of suitable promoters, to prepare a nucleic acid construct of the present invention.

Certain regulatory sequences may also be incorporated into the expression constructs of the present invention. These include non-transcribed regions of the vector, which interact with host cellular proteins to carry out transcription and translation. Such elements may vary in their strength and specificity. Depending on the vector system and host utilized, any number of suitable transcription and/or translation elements, including constitutive, inducible, and repressible promoters, as well as minimal 5′ promoter elements may be used.

A constitutive promoter is a promoter that directs constant expression of a gene in a cell. Examples of some constitutive promoters that are widely used for inducing expression of heterologous polynucleotides include the ADH1 promoter for expression in yeast, those derived from any of the several actin genes, which are known to be expressed in most eukaryotic cell types, and the ubiquitin promoter, which is the promoter of a gene product known to accumulate in many cell types. Examples of constitutive promoters for use in mammalian cells include the RSV promoter derived from Rous sarcoma virus, the CMV promoter derived from cytomegalovirus, β-actin and other actin promoters, and the EF1α promoter.

Also suitable as a promoter in the plasmids of the present invention is a promoter that allows for external control over the regulation of gene expression. One way to regulate the amount and the timing of gene expression is to use an inducible promoter. Unlike a constitutive promoter, an inducible promoter is not always optimally active. An inducible promoter is capable of directly or indirectly activating transcription of one or more DNA sequences or genes in response to an inducing agent (or inducer). Some inducible promoters are activated by physical means, such as the heat shock promoter (HSP), which is activated at certain temperatures. Other promoters are activated by a chemical means, for example, IPTG. Other examples of inducible promoters include the metallothionine promoter, which is activated by heavy metal ions, and hormone-responsive promoters, which are activated by treatment of certain hormones. In the absence of an inducer, the nucleic acid sequences or genes under the control of the inducible promoter will not be transcribed or will only be minimally transcribed. Promoters of the nucleic acid construct of the present invention may be either homologous (derived from the same species as the host cell) or heterologous (derived from a different species than the host cell).

Once the nucleic acid construct of the present invention has been prepared, it may be incorporated into a host cell. This is carried out by transforming or transfecting a host or cell with a plasmid construct of the present invention, using standard procedures known in the art, such as described by Sambrook et al., Molecular Cloning: A Laboratory Manual, Third Edition, Cold Spring Harbor: Cold Spring Harbor Laboratory Press, New York (2001). Suitable hosts and cells for the present invention include, without limitation, bacterial cells, virus, yeast cells, insect cells, plant cells, and mammalian cells, including human cells, as well as any other cell system that is suitable for producing a recombinant protein. Exemplary bacterial cells include, without limitation, E. coli and Mycobacterium sp. Exemplary yeast hosts include without limitation, Pischia pastoris, Saccharomyces cerevisiae, and Schizosaccharomyces pombe. Methods of transformation or transfection may result in transient or stable expression of the genes of interest contained in the plasmids. After transformation, the transformed host cells can be selected and expanded in suitable culture. Transformed cells are first identified using a selection marker simultaneously introduced into the host cells along with the nucleic acid construct of the present invention. Suitable markers include markers encoding for antibiotic resistance, such as resistance to kanamycin, gentamycin, ampicillin, hygromycin, streptomycin, spectinomycin, tetracycline, chloramphenicol, and the like. Any known antibiotic-resistance marker can be used to transform and select transformed host cells in accordance with the present invention. Cells or tissues are grown on a selection medium containing an antibiotic, whereby generally only those transformants expressing the antibiotic resistance marker continue to grow. Additionally, or in the alternative, reporter genes, including, but not limited to, β-galactosidase, β-glucuronidase, luciferase, green fluorescent protein (GFP) or enhanced green fluorescent protein (EGFP), may be used for selection of transformed cells. The selection marker employed will depend on the target species.

To obtain the collectin protein, such as SP-A or SP-D, expression is induced if the coding sequence is under the control of an inducible promoter. To isolate the protein, the host cell carrying an expression vector is propagated, homogenized, and the homogenate is centrifuged to remove bacterial debris. Collectins may be purified using standard anion exchange chromatography. Oberley, R. E. et al., Am J Physiol Lung Cell Mol Physiol 287: L296-306 (2004). Alternative methods of protein purification may be used as suitable. See J. E. Coligan et al., eds., Current Protocols in Protein Science (John Wiley & Sons, 2003). Upon obtaining the substantially purified recombinant protein, the protein may be administered to prevent or treat a gastrointestinal infection as described herein.

Yet another alternative means of achieving the benefits of collectins in preventing or treating a gastrointestinal infection is to increase the expression of endogenous collectins such as SP-A and/or SP-D. Endogenous collectin is any collectin protein that is expressed by the mammal itself and not administered exogenously. Humans are known to produce SP-A in their intestine. Rubio et al., J Biol Chem 270: 12162-12169 (1995); Lin et al., Ped Path Mol Med, 20: 367-286 (2001). Increasing the expression of the a collectin gene such as SP-A gene or otherwise increasing the concentration of a collectin protein above its wild-type levels would provide additional protective effects. Agents suitable for use in the present methods include glucocorticoids, dibutyryladenosine 3′5′-cyclic monophosphate, and keratinocyte growth factor.

### Example

The present example demonstrates that mice lacking the SP-A gene (SP-A null) delivered to similarly SP-A null mothers, died at a higher rate when exposed to environmental bacteria when compared to similar newborn SP-A null mice delivered to mothers that produce SP-A. Additionally, mouse pups that produce their own SP-A (SP-A heterozygotes) or are administered exogenous SP-A have improved survival compared to SP-A null animals when reared by mothers that do not produce SP-A. Consequently, SP-A compositions are useful to prevent or treat a gastrointestinal infection in mammals.

The present example also demonstrates that SP-D is present in lactating mammary tissues and milk, indicating that SP-D production by the mother and secretion into her milk may be a mechanism where by milk protects newborns from common infections. As such, administering isolated SP-D to a mammal having a gastrointestinal infection or at risk for a gastrointestinal infection is predicted to protect against the enteric infection.

### Materials and Methods

Animals. Male and female C3HeB/FeJ mice from Jackson Laboratories (Bar Harbor, Me.) and Swiss Black mice from Taconic (Hudson, N.Y.) were purchased at 6 weeks of age to create breeding colonies. SP-A null mice on a C3H/Hen background were provided for these studies by Dr. F. McCormack (University of Cincinnati). These mice were derived from SP-A null mice originally produced on a Swiss Black genetic background. Both the C3HeB/FeJ and C3H/Hen mice are sub-strains of C3H mice and have been shown to differ from each other by only a single nucleotide polymorphism out of 1638 examined. The SP-D null mice on the Swiss Black background were generously provided by Dr. J. Whitsett (University of Cincinnati) and were compared to wild type Swiss Black mice (Taconic). All mice were housed in isolation cubicles with micro-isolator lids on individual cages. For all studies, mice that served as controls were maintained within the same isolation cubicle in individual cages adjacent to the experimental mice during the same time period. All pups were born and allowed to mature in the presence of both parents in either the control or corn dust bedding environments. Pups were weaned at 21 days of age. Mice were provided food and water ad libitum. All procedures were performed according to protocols approved by the Animal Care and Use Committee at the University of Iowa.

Environmental exposure model. In order to create a non-hygienic environment rich in organic microbes, breeding pairs and their litters were exposed to an organic dust used as bedding material, as described in Ref. (15). Corn dust collected from the drying system at a local grain elevator during the month of October was used to create the non-hygienic environment. Two batches of corn dust were used in the experiments. Batch A corn dust was used for experimental exposures from 2003-2005. Batch B corn dust was used for experimental exposures from 2005-2007. The control environment for all experiments consisted of cellulose fiber bedding, Cellu-Dri (Shepherd Specialty Papers, Kalamazoo, Mich.).

Endotoxin measurements. The endotoxin contents of the bedding and purified human SP-A were determined by the kinetic chromogenic Limulus amebocyte lysate (LAL) assay (Whittaker Bioproducts, Walkersville, Md.) as previously reported (63). The endotoxin content of the purified SP-A was 0.012 ng/μg protein.

Necropsy and histological examination. Necropsies from animals that were euthanized with an overdose of isoflurane when they appeared “critically ill” (dehydrated with reduced spontaneous movement and a distended abdomen) were photographed. Histological examination of the lower respiratory tract and gastrointestinal tissues was performed on mice that were ˜24 h of age. The gastrointestinal system, i.e. stomach to distal large bowel, was dissected en block, then placed into fixative (zinc-formalin or 10% formalin) for 7 days. The right heart was then flushed with ice cold PBS and the lungs inflated with fixative via the trachea at a pressure of 25 cm H₂O (15). All tissues were embedded in paraffin and 5 μm thick sections were mounted onto glass slides, and then stained with hematoxylin and eosin.

Bacterial cultures of the bedding and animals. Bacterial identification and quantification in the bedding materials was performed using standard microbiological techniques. Using sterile technique, peritoneal fluid, blood, and minced lung were collected on a sterile culture swab (BBL CultureSwab, Beckton, Dickinson and Co., Sparks, Md.) and submitted for bacterial identification. Bacteria were identified by either the Clinical Microbiology Laboratory or the University Hygienic Laboratory at the University of Iowa.

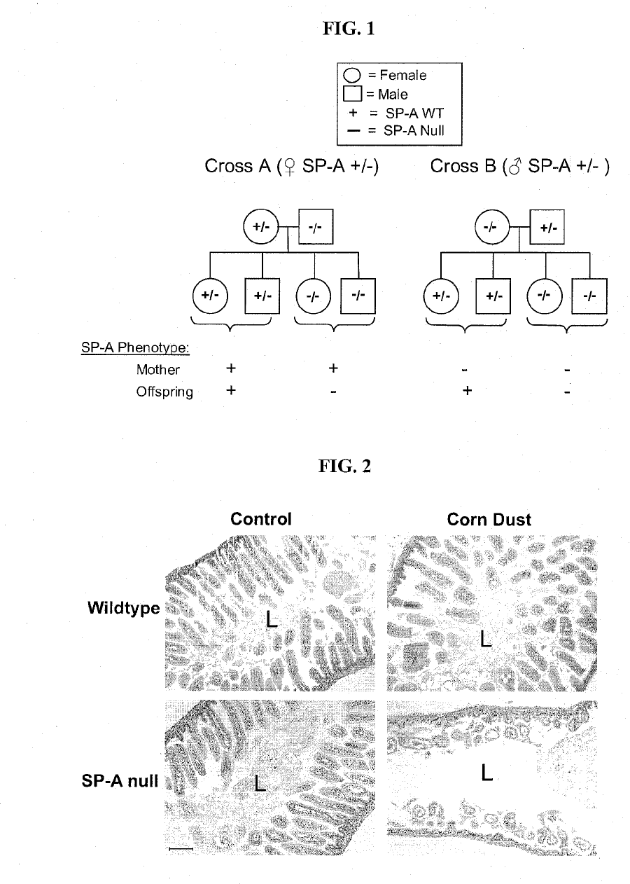

Heterozygous breeding and newborn survival. Crossing C3HeB/FeJ (SP-A +/+) mice with C3H/Hen (SP-A −/−) mice allowed us to generate SP-A heterozygous breeder mice. The heterozygous breeders were then paired with SP-A null breeders according to the strategy shown in FIG. 1. Using the heterozygous breeding strategies ensured that the SP-A gene mutation was controlled for while the C3H sub-strain backgrounds were randomly mixed. Two different crosses were performed. In the first cross (A), the female was heterozygous for SP-A and the male was SP-A null. In the second cross (B), the female was SP-A null and the male was heterozygous for SP-A. Since the progeny of these crosses are either SP-A null (50%) or SP-A heterozygous (50%), there are 4 maternal/neonate outcomes, which are indicated at the bottom of FIG. 1. Throughout this portion of the study, all breeding pairs and their offspring were observed 2-3 times/day. The total number of pregnancies was defined as the number of pregnancies that resulted in the birth of live pups. Only pups that were alive at the time of birth were included in the data collection.

SP-A genotyping. Genotyping for all breeders and their offspring was performed on DNA isolated from tail clips or ear punches using tail lysis buffer (Viagen Biotech, Los Angeles, Calif.). PCR primers amplified the SP-A DNA sequence that had been deleted to create the mutated SP-A null mice (30). The SP-A primers amplified a region that includes portions of SP-A exon 3-4 and intron 3 (forward: 5′ GCAGAGATGGGAGAGATGGTATCAA 3′ (SEQ ID NO: 7) and reverse: 5′ ATGGACCTCCATTAGCATGTGGGA 3′ (SEQ ID NO: 8)). PCR primers were also used to amplify the neomycin insert placed into the SP-A gene (forward: 5′ TGAATGAACTGCAGGACGAG 3′ (SEQ ID NO: 9) and reverse: 5′ ATACTTTCTCGGCAGGAGCA 3′ (SEQ ID NO:10)) in the SP-A null mice. The two PCRs amplified the wild type and mutated SP A genes, respectively. The DNA template was denatured at 94° C., annealed at 60° C. and extension was performed at 74° C. for 25 cycles. PCR products were electrophoresed on a 1.0% agarose gel and photographed.

SP-A and SP-D mRNA determination. Real time RT-PCR for SP-A mRNA was performed on lactating and non-lactating mammary tissues and newborn intestinal tissues. RNA was isolated using Trizol reagent (InVitrogen, Carlsbad, Calif.). A one-step RT-PCR reaction was performed using the SuperScript III Platinum One-Step qRT-PCR system (InVitrogen). For SP-A, the samples were analyzed using FAM-labeled SP-A and 18s rRNA (house-keeping gene) primers (TaqMan® Gene Expression Assays, Applied Biosciences, Foster City, Calif.) and a Stratagene Mx3000P instrument. For SP-D, the samples were analyzed by real-time RT-PCR using FAM-labeled SP-D and GAPDH primers. The cycle thresholds (CT) for both SP-A mRNA and 18s mRNA were used to determine relative SP-A gene expression using the 2^{-ΔΔCT} calculation where the GI tract was used as the reference value (38). The lowest limit of detection for the CT was 40 cycles. The 2^{-ΔΔCT} calculation is as follows: ΔCT=CT SP-A−CT 18s mRNA and using the GI tract as the reference value the ΔΔCT=ΔCT test tissue−ΔCT GI tract. The cycle threshold (CT) method was also used to calculate the relative levels of SP-D RNA and GAPDH RNA. Briefly, ΔCT=CT SP-D−CT GAPDH, then the ΔCT calculation was converted to relative gene expression using the 2^{-ΔΔCT calculation. The average lung} 2^{-ΔΔCT} was made equal to 100+/−SEM to allow for percentage comparison to the mammary gland.

Surfactant protein-A purification and administration. The method of isolating and purifying SP-A from lavage fluid obtained from alveolar proteinosis patients has previously been described (49). Briefly, the lavage material was pelleted and delipidated with isopropyl ether and 1-butanol. The aqueous phase was collected and precipitated with 100% ethanol. The precipitated pellet was re-suspended in 20 mM KH₂PO₄ and further purified using an Affi-gel Blue column (Bio-Rad, Hercules, Calif.). The column flow through was dialyzed against distilled H₂O and the protein concentration was determined by a Bradford assay (Bio Rad). The purity of the SP-A was assessed by electrophoresis on a polyacrylamide gel followed by staining with Coomassie blue (GelCode Blue stain, Pierce, Rockford, Ill.). Purified human SP-A was stored at −80° C. until used. The protein was diluted in sterile PBS just prior to administration. Newborn mice received purified human SP-A (5 μg/5 μl) delivered p.o. twice in the first 24 h of life via a flexible gel loading tip and pipette. This dose lies within the dosing range used in murine studies of pulmonary infection (35, 39). Sham fed littermates received 5 μl sterile PBS via the same delivery technique.

Statistical Evaluation. Log rank analysis was used to determine the relationships between the different outcomes presented in Kaplan-Meier survival curves (SigmaStat, version 3.0). A Chi-squared test was used to evaluate the genetic segregation of the SP-A gene mutation. For all other statistical comparisons, Student's t-test was performed. A “p” value of <0.05 was considered significant. All data are presented as the mean plus or minus the standard error of the mean (SEM).

Immunohistochemistry for SP-D. Formalin-fixed, paraffin-embedded breast tissue samples from two patients were obtained from the Pathology Department at the University of Iowa. The human tissues and the murine mammary tissues were sectioned (5 μm thickness) and mounted onto glass slides for immunohistochemical staining. The primary antibody was a rabbit anti-human SP-D antibody (Chemicon, Temecula, Calif.), which detects both human and murine SP-D. The immunostaining protocols followed have been described previously. Negative controls used non-immune rabbit IgG (Cappel, Irvine, Calif.) in place of the primary antibody, and at the same concentration as primary antibody. Photomicrographs were obtained using a Spot Jr. Digital camera (Sterling Heights, Mich.).

Immunoblotting for SP-D. Human milk was obtained from the Mother's Milk Bank at the Children's Hospital of Iowa. Human and murine milk samples were stored at −80° C. until all of the samples to be analyzed were ready for processing. The frozen milk was thawed on ice, skimmed by centrifugation 14,000×g at 4° C. for 10 min. The proteins present in 30 μl skimmed human milk or 100 μg mammary gland proteins in a reducing sample buffer were separated by electrophoresis in a 15% Tris-HCL polyacrylamide gel under reduced conditions. The separated proteins were then electrophoretically transferred to a Trans-Blot Transfer Medium Membrane (Bio-Rad). Membranes were blocked by overnight incubation at 4° C., in a solution of 7% non-fat dry milk in TNT (0.02M Tris, 0.15M NaCl, 0.01% Tween 20). The membrane was then incubated for 1 h at room temperature with rabbit anti-human SP-D antibody (Chemicon) (diluted 1:1000 in blocking solution), and this was followed by three rinses, for 15 min each, in TNT buffer. Membranes were subsequently incubated for 1 h at room temperature in a secondary antibody conjugated to horseradish peroxidase (Cappel), (diluted 1:10,000 in blocking solution), and then washed three times in TNT buffer. Next, they were exposed to ECL Western Blotting Detection Reagents (Amersham Biosciences, England) for 1 min before exposure to Classic Blue Sensitive X-ray film (Midwest Scientific, St. Louis, Mo.) for 30 seconds.

### Experimental Results

Environmental Exposures. Two batches of corn dust were used in the following experiments. Batch A corn dust contained 57 times more endotoxin than the control bedding. Consistent with those findings, the average endotoxin content in the second corn dust batch (B) was 259 to +/−82 EU/mg (n=4 measurements) while the control bedding material contained significantly lower levels of endotoxin (p<0.001), averaging 11.1 EU/mg+/−3.3. Mice living in cage bedding does not contribute to environmental endotoxin levels. Thus, in our exposure model, the only significant contribution towards environmental endotoxin levels was from the bedding materials (15). The variety of bacteria and fungi identified and quantified in the corn dust bedding are listed in Table 9. In comparison, only 2 morphologies of Bacillus sp. were grown from the control cellulose bedding material.

TABLE 9
Bacterial Identification in Corn Dust Bedding Material

| | Identification |
|---|---|
| Batch A: | Klebsiella oxytoca, Klebsiella pneumonia, Enterobacter |
| | sp., Pseudomonas putida, Staphylococcus sp., Proteus |
| | sp., Enterococcus sp., Bacillus sp. (not anthrasis), and |
| | Rhizopus. |
| Batch B: | Klebsiella pneumonia, Microbacterium sp., Oerskovia |
| | sp., Bacillus sp. (including B. cereus), Chryseobacterium |
| | sp., Enterobacter sp., other gram negative rods, |
| | Acremonium sp., Mucor sp., Cladosporium sp., and |
| | Aspergillus sp. |

The corn dust environment reduces litter size in wild type mice. C3HeB/FeJ mice were bred in the corn dust bedding environment in order to create a perinatal immune stimulus (15). An analysis of the litter sizes produced by breeding pairs exposed to corn dust bedding (batch A, Table 9) versus those exposed to control bedding showed there was a significant reduction in the number of pups/litter at the time of weaning (day of life 21) for animals exposed to corn dust bedding when compared to those exposed to control bedding (2.93+/−0.37 vs. 5.34+/−0.43, respectively, p=<0.001). This translates to a ˜45% reduction in litter size at the time of weaning as a result of exposure to the corn dust environment.

SP-A is critical for newborn survival in the corn dust environment. SP-A null, SP-D null and their respective control wild type mice were bred into corn dust (batch A, Table 9) or control bedding. Each of the SP-A and SP-D null breeding pairs produced at least 2 litters of pups. The litter size at weaning, the number of pregnancies, and adult breeder deaths were recorded during a 5-month period (Table 10). The C3HeB/FeJ wild type mice have smaller litters in the corn dust bedding when compared to the litter size in the control bedding exposed animals (p<0.05). Survival data for adult breeders housed in both environmental conditions are presented as the number of deaths/total number of adult animals in each experimental condition (Table 10). Litter size is defined as the number of pups at the time of weaning (21 days of life). The data are the mean+/−SEM, where * p=0.012, SP-A +/+ mice, corn dust versus control bedding; ** p<0.001, SP-A −/− mice, control vs corn dust bedding. Among the 4 SP-A null breeding pairs maintained in the corn dust environment, there were 10 pregnancies that resulted in the birth of live pups at 19.5-20 days gestation. While all 10 pregnancies produced live pups, none of these pups survived past 48 h of life (Table 10). In contrast, the SP-A null breeding pairs maintained in control bedding produced ˜5 pups/litter in 24 pregnancies. Similar to the C3HeB/FeJ wild type mice, the Swiss Black wild type mice also produced smaller litters in the corn dust bedding than in control bedding, however, the difference did not reach statistical significance (Table 10). During the 5-month time period only 1 out of 28 adult mice exposed to corn dust bedding died. This animal was a C3HeB/FeJ wild type mouse. Finally, to ensure that the SP-A null breeders could successfully rear pups, one pair maintained in corn dust was placed into control bedding. This breeding pair had previously delivered 2 litters in the corn dust bedding environment, in which all of the pups died. Placing this breeding pair into the control bedding allowed them to have 3 subsequent litters of pups with an average of 3.5+/−2.1 pups/litter at weaning.

TABLE 10
Effect of corn dust on adult and newborn survival
A. C3H

| | Genotype | | | |
|---|---|---|---|---|
| | SP-A+/+ | | SP-A−/− | |
| | | Corn | | Corn |
| Bedding | Control | Dust | Control | Dust |
| # of | 17 | 14 | 24 | 10 |
| Pregnancies | | | | |
| adult deaths/ | 0/20 | 1/8 | 0/10 | 0/8 |
| total | | | | |
| pups/litter | 5.8 +/− 0.7 | 2.8 +/− 0.6* | 5.0 +/− 0.4 | 0** |
| (mean +/− | | | | |
| SEM) | | | | |

B. Swiss Black

| | Genotype | | | |
|---|---|---|---|---|
| | SP-D+/+ | | SP-D−/− | |
| | | Corn | | Corn |
| Bedding | Control | Dust | Control | Dust |
| # of | 12 | 9 | 14 | 7 |
| Pregnancies | | | | |
| adult deaths/ | 0/6 | 0/6 | 0/6 | 0/6 |
| total | | | | |
| pups/litter | 8.5 +/− 1.0 | 6.1 +/− 1.2 | 8.7 +/− 0.5 | 7.9 +/− 0.7 |
| (mean +/− | | | | |
| SEM) | | | | |

SP-A null pups survive in a sterile but high endotoxin environment. Autoclaving the corn dust (batch A, Table 8) for 30 min at 120° C. rendered it sterile. The autoclaved corn dust, however, remained high in endotoxin content (170 EU/mg) when compared to control bedding. Three SP-A null breeding pairs were placed in the autoclaved corn dust bedding, and six litters were born with an average of 2.67+/−1.63 pups/litter at the time of weaning. Thus, sterilizing the corn dust allowed the SP-A null offspring to survive until weaning. The litter size for the SP-A null breeding pairs maintained in autoclaved corn dust did not return to the baseline values (5.0±0.4 pups/litter) observed when SP-A null mice were born into control, semi-sterile bedding (p<0.05).

Gastrointestinal pathology in newborn SP-A null mice exposed to corn dust bedding. Necropsy and histological examination of the newborn SP-A null mice born in the corn dust bedding were performed and the results compared to SP-A null mice born in control bedding and also to wild type mice born in both environments. The pups appeared critically ill (i.e., gasping respirations, bloated abdomen, with signs of dehydration) at the time they were euthanized. Gross abnormalities were observed in the intestines while other abdominal and thoracic organs appeared normal. These characteristics were present in critically ill SP-A null mice exposed to corn dust, but were not observed in age matched wild type mice exposed to the same environment (data not shown). The gross morphology of the gastrointestinal (GI) tract, from the stomach to the distal large bowel, in a healthy 24 h old wild type mouse was compared to that of GI tracts from two SP-A null mice reared in the corn dust bedding. The stomach and proximal small bowel of the wild type mice were white and full of milk whereas the SP-A null mice had empty bile colored stomachs and proximal small bowels. Histological examination of lung tissues from 24 h old wild type and SP-A null mice reared in both environments demonstrated no difference in lung structure between the genetic strains or the environmental conditions. However, blood congestion was consistently observed in the lungs of SP-A null mice reared in corn dust who appeared to be critically ill at 24 h of age when compared to the lungs of wild type mice reared in both environmental conditions and SP-A null mice reared in control bedding. In the GI tract, the most consistent structural difference observed in the small bowel of SP-A null mice exposed to corn dust as compared to SP-A null mice reared in control bedding or to wild type mice reared in either condition was a marked dilation of the intestinal lumen (FIG. 2). Histological differences were also observed in the GI tracts of 24 h old SP-A null mice exposed to corn dust when compared to those of SP A null mice reared in control bedding. Specifically, in the stomachs of SP-A null mice exposed to corn dust, we observed neutrophil accumulation and sloughing epithelial cells with condensed nuclei. The proximal small bowel of critically ill SP-A null pups reared in corn dust bedding had more readily detectable neutrophils within and marginated along the walls of moderately congested gastric blood vessels.

Bacteria isolated from critically ill newborn SP-A null mice. Gross examination of critically ill SP-A null mice born and reared in corn dust bedding frequently revealed abdominal distention. During necropsy, fluid was often noted in the peritoneal space, a finding never observed in healthy newborn mice. Bacterial cultures of peritoneal fluid obtained from critically ill SP-A null pups reared in corn dust were obtained using a sterile culture swab and sterile technique (Table 11). Mice born into corn dust bedding were described as critically “ill” with at least 2 of the following findings: distended abdomen, dehydration, decreased spontaneous movements, or abnormal respiration pattern (Y indicates yes and N indicates no). The source of the bacterial culture is described and culture results are indicated by the bacteria identified or as culture negative (O). Bacillus sp. (not anthracis) was isolated from the peritoneum in 3 of 5 μl SP-A null pups, while Entercoccus sp. was isolated once. In contrast, 3 blood cultures and 1 lung tissue culture from these animals were negative for bacteria.

TABLE 11
Bacterial Culture Data from SP-A Null Newborn Mice

| III appearing | Source | Culture results |
|---|---|---|
| Y | peritoneum | Bacillus sp, Enterococcus sp. |
| | blood | Ø |
| Y | peritoneum | Ø |
| N | peritoneum | Ø |
| Y | peritoneum | Bacillus sp. |
| | blood | Ø |
| Y | peritoneum | Bacillus sp. |
| | lung | Ø |
| Y | peritoneum | Ø |
| | blood | Ø |

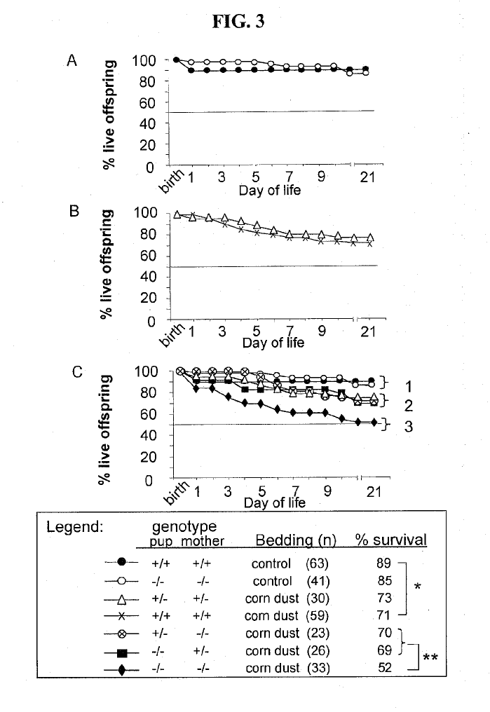

Effects of maternal and neonatal SP-A on newborn survival. Survival data from birth to 21 days of life were collected for offspring born to heterozygous breeding pairs (FIG. 1) or wild type breeding pairs maintained corn dust bedding (batch B, Table 9) or control bedding. Tissue samples for genotyping were available for 93% of all live born offspring and correlated with survival. The anticipated SP-A genotype of the pups generated according to the heterozygous breeding in FIG. 1 was 50%−/− and 50%+/−. The offspring genotype data from the heterozygous breeding closely followed Mendel's law of segregation (p=0.789, Chi-squared test with 1 degree of freedom), with 47% of the offspring having the genotype SP-A +/− and 53% having a SP-A −/− genotype. Of note, corn dust batch B used for this and the remainder of our studies was not as lethal to the SP-A null pups as was batch A. The survival of wild type and SP-A null pups reared in the control bedding did not differ (FIG. 3A). Exposure to corn dust significantly reduced wild type pup survival (*p=0.022) compared to the survival of wild type pups reared in control bedding (71% vs. 89%, respectively, FIG. 3, panels A and B). There was no difference in pup survival when comparing SP-A heterozygous pups born to heterozygous mothers to wild type pups born to wild type mothers in the corn dust bedding (FIG. 3B). However, a significant reduction in pup survival (p 0.048) was found in SP-A null pups born to SP-A null mothers when compared to the survival of SP-A heterozygous pups born to SP-A heterozygous mothers (52% vs 73%, respectively), reared in the same corn dust environment. Finally, if either the mother or the pup produced SP-A, the pups' survival in the corn dust environment was significantly improved (**p=0.048) when compared pup survival when there was a lack of SP-A in both mother and pup (FIG. 3C).

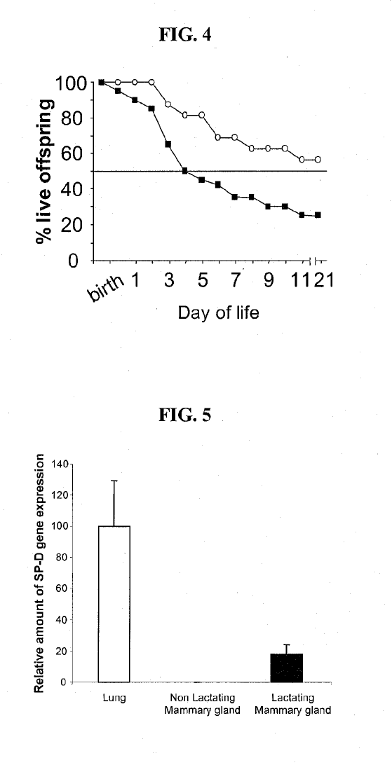

Oral purified human SP-A improves survival of SP-A null newborn pups. SP-A null pairs were allowed to breed in the corn dust bedding (Table 9, batch B). Newborn pups were fed purified human SP-A (5 μg) twice during the first 24 h of life. Littermates fed sterile PBS (diluent) in place of the SP-A served as the control group. At 5 days of life, the SP-A null newborn pups who received enteral SP-A had a significant improvement in their survival rate, when compared to SP-A null pups that received PBS (81.3% vs. 45.0%, respectively, p=0.027, by log rank analysis with n=16 and 20 pups/group respectively, FIG. 4). At day of life 21, the positive effect of SP-A treatment on SP-A null pup survival in corn dust bedding persisted (p=0.035) when compared to SP-A null pups who did not receive SP-A. Thus, the administration of SP-A is effective at improving pup survival, and compositions comprising SP-A may be used in the prevention or treatment of gastrointestinal infections.

SP-A and SP-D mRNA. Semi-quantitative real-time RT-PCR on murine mammary tissues obtained from lactating (between 24 hours and 12 days post partum) and non-lactating (nulliparous) females were analyzed for SP-A and SP-D gene expression. Of the 5 lactating mammary samples assayed, none demonstrated any SP-A RNA, Conversely, analyzing these tissues for SP-D revealed a lactation-dependent increase in SP-D gene expression (FIG. 5). SP-D total RNA was present in all lactating mammary gland samples. On average the levels were ˜20% of that observed in the lung. Only one of the three non-lactating mice had a measurable CT, all-be-it just within the level of detection, while the other two samples had immeasurable levels of SP-D expression. Therefore, SP-D RNA was virtually undetectable in non-lactating mammary tissue, and present in lactating mammary tissue.

SP-D localization by immunohistochemistry. Both lactating and non-lactating mammary tissues from human and mouse were immunostained for SP-D protein using rabbit anti-human antibodies with interspecies cross-specificity. In both the human and murine non-lactating tissue, the surrounding ductal epithelial cells failed to stain for SP-D protein. In the non-lactating human sample, areas of positive, antibody-specific (vs negative control), staining were observed within the lumina of the mammary ducts. In contrast, lactating mammary tissue of both species demonstrated intense cytoplasmic SP-D immunostaining of the mammary gland epithelial cells. SP-D staining was also prominent in the secreted milk present in the lumina of the ducts.

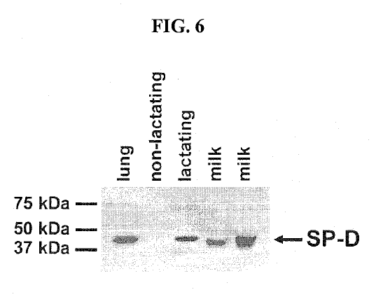

SP-D protein by Immunoblot analysis. Western blot analysis also revealed the presence of SP-D protein in lactating murine mammary tissue, as well as in murine milk (FIG. 6). The molecular weight of the immunoreactive band was about 43 kDa. In contrast, SP-D protein was not found in the non-lactating murine mammary tissue. The function of SP-D in mammary tissue and milk may be two-fold. Milk-borne SP-D may provide immunoprotection to the newborn, and it may also help to prevent infection (mastitis) in the mother. As such, administering isolated SP-D to a mammal having a gastrointestinal infection or at risk for a gastrointestinal infection is predicted to protect against the enteric infection.

All publications, patent applications, issued patents, and other documents referred to in this specification are herein incorporated by reference as if each individual publication, patent application, issued patent, or other document was specifically and individually indicated to be incorporated by reference in its entirety.

It is not intended that the present invention be limited to the described embodiments. It is intended that the invention cover all modifications and alternatives which may be included within the spirit and scope of the invention. Consequently, the embodiments described here are to be taken as illustrative, not limiting. Various modifications to the disclosed embodiments will be readily apparent to those skilled in the art, and generic principles may be applied to other embodiments.

Other embodiments are set forth in the following claims.

### REFERENCES

1. Adler, A., G. Gottesman, T. Dolfin, S. Amon, R. Regev, S. Bauer, and I. Litmanovitz. 2005. Bacillus species sepsis in the neonatal intensive care unit. J Infect 51:390-395.
2. Akiyama, J., A. Hoffman, C. Brown, L. Allen, J. Edmondson, F. Poulain, and S. Hawgood. 2002. Tissue distribution of surfactant proteins A and D in the mouse. J Histochem Cytochem 50:993-996.
3. Borron, P. J., E. A. Mostaghel, C. Doyle, E. S. Walsh, M. G. McHeyzer-Williams, and J. R. Wright. 2002. Pulmonary surfactant proteins A and D directly suppress CD3+/CD4+ cell function: evidence for two shared mechanisms. J Immunol 169:5844-5850.
4. Botas, C., F. Poulain, J. Akiyama, C. Brown, L. Allen, J. Goerke, J. Clements, E.
Carlson, A. M. Gillespie, C. Epstein, and S. Hawgood. 1998. Altered surfactant homeostasis and alveolar type II cell morphology in mice lacking surfactant protein D. Proc Natl Acad Sci USA 95:11869-11874.
5. Braidotti, P., C. Cigala, D. Graziani, B. Del Curto, E. Dessy, G. Coggi, S. Bosari, and G. G. Pietra. 2001. Surfactant protein A expression in human normal and neoplastic breast epithelium. Am J Clin Pathol 116:721-728.
6. Brinker, K. G., H. Garner, and J. R. Wright. 2003. Surfactant protein A modulates the differentiation of murine bone marrow-derived dendritic cells. Am J Physiol Lung Cell Mol Physiol 284:L232-241.
7. Condon, J. C., P. Jeyasuria, J. M. Faust, and C. R. Mendelson. 2004. Surfactant protein secreted by the maturing mouse fetal lung acts as a hormone that signals the initiation of parturition. Proc Natl Acad Sci USA 101:4978-4983.
8. Crouch, E., K. Rust, W. Marienchek, D. Parghi, D. Chang, and A. Persson. 1991. Developmental expression of pulmonary surfactant protein D (SP-D). Am J Respir Cell Mol Biol 5:13-18.
9. Crouch, E. C. 1998. Collectins and pulmonary host defense. Am J Respir Cell Mol Biol 19:177-201.
10. DiAngelo, S., Z. Lin, G. Wang, S. Phillips, M. Ramet, J. Luo, and J. Floros. 1999. Novel, non-radioactive, simple and multiplex PCR-cRFLP methods for genotyping human SP-A and SP-D marker alleles. Dis Markers 15:269-281.
11. Doyle, 1. R., H. A. Barr, and T. E. Nicholas. 1994. Distribution of surfactant protein A in rat lung. Am J Respir Cell Mol Biol 11:405-415.
12. From, C., R. Pukall, P. Schumann, V. Hormazabal, and P. E. Granum. 2005. Toxin-producing ability among Bacillus spp. outside the Bacillus cereus group. Appl Environ Microbiol 71:1178-1183.
13. Gaur, A. H., and J. L. Shenep. 2001. The expanding spectrum of disease caused by Bacillus cereus. Pediatr Infect Dis J 20:533-534.
14. George, C. L., H. Jin, C. L. Wohiford-Lenane, M. E. O'Neill, J. C. Phipps, P.
O'Shaughnessy, J. N. Kline, P. S. Thorne, and D. A. Schwartz. 2001. Endotoxin responsiveness and subchronic grain dust-induced airway disease. Am J Physiol Lung Cell Mol Physiol 280:L203-213,
15. George, C. L., M. L. White, K. Kulhankova, A. Mahajan, P. S. Thorne, J. M. Snyder, and J. N. Kline. 2006. Early exposure to a nonhygienic environment alters pulmonary immunity and allergic responses. Am J Physiol Lung Cell Mol Physiol 291:L512-522.
16. George, C. L., M. L. White, M. E. O'Neill, P. S. Thorne, D. A. Schwartz, and J. M. Snyder. 2003. Altered surfactant protein A gene expression and protein metabolism associated with repeat exposure to inhaled endotoxin. Am J Physiol Lung Cell Mol Physiol 285:L1337-1344.
17. Girisch, M., M. Ries, M. Zenker, R. Carbon, R. Rauch, and M. Hofbeck. 2003. Intestinal perforations in a premature infant caused by Bacillus cereus. Infection 31:192-193.
18. Goss, K. L., A. R. Kumar, and J. M. Snyder. 1998. SP-A2 gene expression in human fetal lung airways. Am J Respir Cell Mol Biol 19:613-621.
19. Hilliard, N. J., R. L. Schelonka, and K. B. Waites. 2003. Bacillus cereus bacteremia in a preterm neonate. J Clin Microbiol 41:3441-3444.
20. Holmskov, U., S. Thiel, and J. C. Jensenius. 2003. Collections and ficolins: humoral lectins of the innate immune defense. Annu Rev Immunol 21:547-578.
21. Jack, D. L., J. Cole, S. C. Naylor, R. Borrow, E. B. Kaczmarski, N. J. Klein, and R. C. Read. 2006. Genetic polymorphism of the binding domain of surfactant protein-A2 increases susceptibility to meningococcal disease. Clin Infect Dis 43:1426-1433.
22. Karinch, A. M., D. E. deMello, and J. Floros. 1997. Effect of genotype on the levels of surfactant protein A mRNA and on the SP-A2 splice variants in adult humans. Biochem J 321 (Pt 1):39-47.
23. Katyal, S. L., G. Singh, and J. Locker. 1992. Characterization of a second human pulmonary surfactant-associated protein SP-A gene. Am J Respir Cell Mol Biol 6:446-452.
24. Kawasaki, N., T. Kawasaki, and I. Yamashina. 1989. A serum lectin (mannan-binding protein) has complement-dependent bactericidal activity. J Biochem (Tokyo) 106:483-489.
25. Khoor, A., M. E. Gray, W. M. Hull, J. A. Whitsett, and M. T. Stahlman. 1993. Developmental expression of SP-A and SP-A mRNA in the proximal and distal respiratory epithelium in the human fetus and newborn. J Histochem Cytochem 41:1311-1319.
26. Khubehandani, K. R., and J. M. Snyder. 2001. Surfactant protein A (SP-A): the alveolus and beyond. Faseb J 15:59-69.
27. Kim, J. K., S. S. Kim, K. W. Rha, C. H. Kim, J. H. Cho, C. H. Lee, J. G. Lee, and J. H. Yoon. 2007. Expression and localization of surfactant proteins in human nasal epithelium. Am J Physiol Lung Cell Mol. Physiol.
28. Koible, K., J. Lu, S. E. Mole, S. Kaluz, and K. B. Reid. 1993. Assignment of the human pulmonary surfactant protein D gene (SFTP4) to 10q22-q23 close to the surfactant protein A gene cluster. Genomics 17:294-298.
29. Korfhagen, T. R., M. D. Bruno, S. W. Glasser, P. J. Ciraolo, J. A. Whitsett, D. L. Lattier, K. A. Wikenheiser, and J. C. Clark. 1992. Murine pulmonary surfactant SP-A gene: cloning, sequence, and transcriptional activity. Am J Physiol 263:L546-554.
30. Korfiagen, T. R., M. D. Bruno, G. F. Ross, K. M. Huelsman, M. Ikegami, A. H. Jobe, S. E. Wert, B. R. Stripp, R. E. Morris, S. W. Glasser, C. J. Bachurski, H. S. Iwamoto, and J. A. Whitsett. 1996. Altered surfactant function and structure in SP-A gene targeted mice. Proc Natl Acad Sci USA 93:9594-9599.
31. Kuzmenko, A. I., H. Wu, S. Wan, and F. X. McCormack. 2005. Surfactant protein A is a principal and oxidation-sensitive microbial permeabilizing factor in the alveolar lining fluid. J Biol Chem 280:25913-25919.
32. LeVine, A. M., M. D. Bruno, K M. Huelsman, G. F. Ross, J. A. Whitsett, and T. R. Korfhagen. 1997. Surfactant protein A-deficient mice are susceptible to group B streptococcal infection. J Immunol 158:4336-4340.
33. LeVine, A. M., J. Elliott, J. A. Whitsett, A. Srikiatkhachorn, E. Crouch, N. DeSilva, and T. Korfhagen. 2004. Surfactant protein-d enhances phagocytosis and pulmonary clearance of respiratory syncytial virus. Am J Respir Cell Mol Biol 31:193-199.
34. LeVine, A. M., J. Gwozdz, J. Stark, M. Bruno, J. Whitsett, and T. Korfhagen. 1999. Surfactant protein-A enhances respiratory syncytial virus clearance in vivo. J Clin Invest 103:1015-1021.
35. LeVine, A. M., K. E. Kurak, J. R. Wright, W. T. Watford, M. D. Bruno, G. F. Ross, J. A. Whitsett, and T. R. Korfhagen. 1999. Surfactant protein-A binds group B streptococcus enhancing phagocytosis and clearance from lungs of surfactant protein-A-deficient mice. Am J Respir Cell Mol Biol 20:279-286.
36. LeVine, A. M., J. A. Whitsett, J. A. Gwozdz, T. R. Richardson, J. H. Fisher, M. S. Burhans, and T. R. Korfhagen. 2000. Distinct effects of surfactant protein A or D deficiency during bacterial infection on the lung. J Immunol 165:3934-3940.
37. Lin, Z., D. deMello, D. S. Phelps, W. A. Koltun, M. Page, and J. Floros. 2001. Both human SP-A1 and Sp-A2 genes are expressed in small and large intestine. Pediatr Pathol Mol Med 20:367-386.
38. Livak, K. J., and T. D. Schmittgen. 2001. Analysis of relative gene expression data using real-time quantitative PCR and the 2(-Delta Delta C(T)) Method. Methods 25:402-408.
39. Madan, T., U. Kishore, M. Singh, P. Strong, H. Clark, E. M. Hussain, K. B. Reid, and P. U. Sarma. 2001. Surfactant proteins A and D protect mice against pulmonary hypersensitivity induced by Aspergillus fumigatus antigens and allergens. J Clin Invest 107:467-475.
40. Madsen, J., A. Kliem, I. Tornoe, K. Skjodt, C. Koch, and U. Holmskov. 2000. Localization of lung surfactant protein D on mucosal surfaces in human tissues. J Immunol 164:5866-5870.
41. Madsen, J., I. Tornoe, O, Nielsen, C. Koch, W. Steinhilber, and U. Holmskov. 2003. Expression and localization of lung surfactant protein A in human tissues. Am J Respir Cell Mol Biol 29:591-597.
42. Mason, R. J., M. C. Lewis, K. E. Edeen, K. McCormick-Shannon, L. D. Nielsen, and J. M. Shannon. 2002. Maintenance of surfactant protein A and D secretion by rat alveolar type II cells in vitro. Am J Physiol Lung Cell Mol Physiol 282:L249-258.
43. Matsuda, N., Y. Hattori, Y. Takahashi, J. Nishihira, S. Jesmin, M. Kobayashi, and S. Gando. 2004. Therapeutic effect of in vivo transfection of transcription factor decoy to NF-kappaB on septic lung in mice. Am J Physiol Lung Cell Mol Physiol 287:L1248-1255.
44. McCormick, S. M., and C. R. Mendelson. 1994. Human SP-A1 and SP-A2 genes are differentially regulated during development and by cAMP and glucocorticoids. Am J Physiol 266:L367-374.
45. Moore, K. J., M. A. D'Amore-Bruno, T. R. Korfhagen, S. W. Glasser, J. A. Whitsett, N. A. Jenkins, and N. G. Copeland. 1992. Chromosomal localization of three pulmonary surfactant protein genes in the mouse. Genomics 12:388-393.
46. Mori, K., N. Kurihara, S. Hayashida, M. Tanaka, and K. Ikeda. 2002. The intrauterine expression of surfactant protein D in the terminal airways of human fetuses compared with surfactant protein A. Eur J Pediatr 161:431-434.
47. Motwani, M., R. A. White, N. Guo, L. L. Dowler, A. I. Tauber, and K. N. Sastry. 1995. Mouse surfactant protein-D. cDNA cloning, characterization, and gene localization to chromosome 14. J Immunol 155:5671-5677.
48. O'Dea, K. P., A. J. Young, H. Yamamoto, J. L. Robotham, F. M. Brennan, and M. Takata. 2005. Lung-marginated monocytes modulate pulmonary microvascular injury during early endotoxemia. Am J Respir Crit. Care Med 172:1119-1127.
49. Oberley, R. E., K. A. Ault, T. L. Neff, K. R. Khubchandani, E. C. Crouch, and J. M. Snyder. 2004. Surfactant proteins A and D enhance the phagocytosis of Chlamydia into THP-1 cells. Am J Physiol Lung Cell Mol Physiol 287:L296-306.
50. Ogasawara, Y., Y. Kuroki, M. Shiratori, H. Shimizu, K. Miyamura, and T. Akino. 1991. Ontogeny of surfactant apoprotein D, SP-D, in the rat lung. Biochim Biophys Acta 1083:252-256.
51. Petkov, P. M., Y. Ding, M. A. Cassell, W. Zhang, G. Wagner, E. E. Sargent, S. Asquith, V. Crew, K. A. Johnson, P. Robinson, V. E. Scott, and M. V. Wiles. 2004. An efficient SNP system for mouse genome scanning and elucidating strain relationships. Genome Res 14:1806-1811.
52. Pikaar, J. C., W. F. Voorhout, L. M. van Golde, J. Verhoef, J. A. Van Strijp, and J. F. van Iwaarden. 1995. Opsonic activities of surfactant proteins A and D in phagocytosis of gram-negative bacteria by alveolar macrophages. J Infect Dis 172:481-489.
53. Premer, D. M., R. Goertz, M. K. Georgieff, M. C. Mammel, and S. J. Schwarzenberg. 2002. Muscle proteolysis and weight loss in a neonatal rat model of sepsis syndrome. Inflammation 26:97-101.
54. Ramet, M., R. Haataja, R. Marttila, J. Floros, and M. Hallman. 2000. Association between the surfactant protein A (SP-A) gene locus and respiratory-distress syndrome in the Finnish population. Am J Hum Genet. 66:1569-1579.
55. Ronnestad, A., T. G. Abrahamsen, S. Medbo, H. Reigstad, K. Lossius, P. I. Kaaresen, I. E. Engelund, L. M. Irgens, and T. Markestad. 2005. Septicemia in the first week of life in a Norwegian national cohort of extremely premature infants. Pediatrics 115:e262-268.
56. Rubio, S., T. Lacaze-Masmonteil, B. Chailley-Heu, A. Kahn, J. R. Bourbon, and R. Ducroc. 1995. Pulmonary surfactant protein A (SP-A) is expressed by epithelial cells of small and large intestine. J Biol Chem 270:12162-12169.
57. Saxon, A., and E. Steiehm. 1989. p. 40-67. In E. Stiehm (ed.), Immunologic Disorders in Infants and Children, 3rd ed. W. B. Saunders, Co., Phildelphia.
58. Schagat, T. L., J. A. Wofford, K. E. Greene, and J. R. Wright. 2003. Surfactant protein A differentially regulates peripheral and inflammatory neutrophil chemotaxis. Am J Physiol Lung Cell Mol Physiol 284:L140-147.
59. Schoeni, J. L., and A. C. Wong. 2005. Bacillus cereus food poisoning and its toxins. Food Prot 68:636-648.
60. Sinchaikul, S., B. Sookkheo, S. Topanuruk, H. F. Juan, S. Phutrakul, and S. T. Chen. 2002. Bioinformatics, functional genomics, and proteomics study of Bacillus sp. J Chromatogr B Analyt Technol Biomed Life Sci 771:261-287.
61. Stahlman, M. T., M. E. Gray, W. M. Hull, and J. A. Whitsett. 2002. Immunolocalization of surfactant protein-D (SP-D) in human fetal, newborn, and adult tissues. J Histochem Cytochem 50:651-660.
62. Sullivan, L. C., C. B. Daniels, I. D. Phillips, S. Orgeig, and J. A. Whitsett. 1998. Conservation of surfactant protein A: evidence for a single origin for vertebrate pulmonary surfactant. J Mol Evol 46:131-138.
63. Thorne, P. S. 2000. Inhalation toxicology models of endotoxin- and bioaerosol-induced inflammation. Toxicology 152:13-23.
64. Tino, M. J., and J. R. Wright. 1999. Surfactant proteins A and D specifically stimulate directed actin-based responses in alveolar macrophages. Am J Physiol 276:L164-174.
65. Tschernig, T., A. S. Debertin, F. Paulsen, W. J. Kleemann, and R. Pabst. 2001. Dendritic cells in the mucosa of the human trachea are not regularly found in the first year of life. Thorax 56:427-431.
66. Tuladhar, R., S. K. Patole, T. H. Koh, R. Norton, and J. S. Whitehall. 2000. Refractory Bacillus cereus infection in a neonate. Int J Clin Pract 54:345-347.
67. Watson, R. S., J. A. Carcillo, W. T. Linde-Zwirble, G. Clermont, J. Lidicker, and D. C. Angus. 2003. The epidemiology of severe sepsis in children in the United States. Am J Respir Crit. Care Med 167:695-701.
68. Wert, S. E., M. Yoshida, A. M. LeVine, M. Ikegami, T. Jones, G. F. Ross, J. H.
Fisher, T. R. Korfhagen, and J. A. Whitsett. 2000. Increased metalloproteinase activity, oxidant production, and emphysema in surfactant protein D gene-inactivated mice. Proc Natl Acad Sci USA 97:5972-5977.
69. Wong, C. J., J. Akiyama, L. Allen, and S. Hawgood. 1996. Localization and developmental expression of surfactant proteins D and A in the respiratory tract of the mouse. Pediatr Res 39:930-937.
70. Wright, J. R. 1997. Immunomodulatory functions of surfactant. Physiol Rev 77:931-962.
71. Wright, J. R. 2005. Immunoregulatory functions of surfactant proteins. Nat Rev Immunol 5:58-68.
72. Wu, H., A. Kuzmenko, S. Wan, L. Schäffer, A. Weiss, J. H. Fisher, K. S. Kim, and F. X. McCormack. 2003. Surfactant proteins A and D inhibit the growth of Gram-negative bacteria by increasing membrane permeability. J Clin Invest 111: 1589-1602.
73. Wu, Y., S. Adam, L. Hamann, H. Heine, A. J. Ulmer, U. Buwitt-Beckmann, and C. Stamme. 2004. Accumulation of inhibitory kappaB-alpha as a mechanism contributing to the anti-inflammatory effects of surfactant protein-A. Am J Respir Cell Mol Biol 31:587-594.

What is claimed is:

1. A composition comprising one or more isolated surfactant-associated proteins and a fluid suitable for oral administration, wherein the surfactant-associated protein is selected from the group consisting of surfactant-associated protein-A (SP-A), surfactant associated protein-D (SP-D), an active fragment of SP-A, an active fragment of SP-D, an active derivative of SP-A, and an active derivative of SP-D, each active fragment or derivative having at least 95% amino acid sequence identity to a SP-A or SP-D protein, and wherein the one or more surfactant-associated proteins are present in a therapeutically effective amount for the prevention or treatment of gastrointestinal infections in mammals.

2. The composition of claim 1, wherein the fluid suitable for oral administration is infant formula or milk.

3. The composition of claim 2, wherein the fluid suitable for oral administration is infant formula.

4. The composition of claim 1, wherein the therapeutically effective amount of one or more surfactant-associated proteins is from about 0.01 mg/kg to about 2 mg/kg.

5. The composition of claim 1, wherein the surfactant-associated protein is human, rat, or bovine SP-A.

6. The composition of claim 5, wherein the human SP-A is SP-A1 or SP-A2.

7. The composition of claim 1, wherein the surfactant associated protein is SP-A1, having an amino acid sequence selected from SEQ ID NO: 3, an active fragment thereof or an active derivative thereof, wherein the active fragment or derivative has at least 95% amino acid sequence identity to SEQ ID NO: 3.

8. The composition of claim 1, wherein the surfactant associated protein is SP-A2, having an amino acid sequence selected from SEQ ID NO: 4, an active fragment thereof or an active derivative thereof, wherein the active fragment or derivative has at least 95% amino acid sequence identity to SEQ ID NO: 4.

9. The composition of claim 1, wherein the SP-A is a heterotrimer of two molecules of SP-A1 and one molecule of SP-A2.

10. A method comprising administering to a mammal having a gastrointestinal infection or at risk for a gastrointestinal infection a therapeutically effective amount of one or more isolated surfactant-associated proteins, wherein the surfactant-associated protein is selected from the group consisting of surfactant-associated protein-A (SP-A), surfactant associated protein-D (SP-D), an active fragment of SP-A, an active fragment of SP-D, an active derivative of SP-A, and an active derivative of SP-D, each active fragment or derivative having at least 95% amino acid sequence identity to an SP-A or SP-D protein.

11. The method of claim 10, wherein the one or more surfactant-associated proteins are administered prior to the onset of infection.

12. The method of claim 10, wherein the one or more surfactant-associated proteins are administered after the onset of infection.

13. The method of claim 10, wherein the gastrointestinal infection is caused by a viral pathogen.

14. The method of claim 10, wherein the gastrointestinal infection is caused by a bacterial pathogen.

15. The method of claim 14, wherein the bacterial pathogen is selected from the group consisting of Klebsiella oxytoca, Klebsiella pneumonia, Enterobacter sp., Clostridium, Pseudomonas putida, E. coli, Group B streptococci, Listeria, Staphylococcus aureus, Salmonella, and Bacillus sp.

16. The method of claim 10, wherein the mammal is a human, rat, cat, dog, cow, pig, mouse, equine, or primate.

17. The method of claim 16, wherein the mammal is a human infant.

18. The method of claim 10, wherein the composition is administered within 7 days of birth.

19. The method of claim 10 wherein the one or more surfactant-associated proteins are administered orally.

20. The method of claim 19 wherein the one or more surfactant-associated proteins are administered with infant formula or milk.


SEQUENCE LISTING (12 sequences)

SEQ ID NO: 1 - DNA, 738 bp, Homo sapiens
atgtggctgtgccctctggccctcaacctcatcttgatggcagcctctggtgctgtgtgcgaagtgaaggacgtttgtgttggaagccctggtatccccggcactcctggatcccacggcctgccaggcagggacgggagagatggtctcaaaggagaccctggccctccaggccccatgggtccacctggagaaatgccatgtcctcctggaaatgatgggctgcctggagcccctggtatccctggagagtgtggagagaagggggagcctggcgagaggggccctccagggcttccagctcatctagatgaggagctccaagccacactccacgactttagacatcaaatcctgcagacaaggggagccctcagtctgcagggctccataatgacagtaggagagaaggtcttctccagcaatgggcagtccatcacttttgatgccattcaggaggcatgtgccagagcaggcggccgcattgctgtcccaaggaatccagaggaaaatgaggccattgcaagcttcgtgaagaagtacaacacatatgcctatgtaggcctgactgagggtcccagccctggagacttccgctactcagacgggacccctgtaaactacaccaactggtaccgaggggagcccgcaggtcggggaaaagagcagtgtgtggagatgtacacagatgggcagtggaatgacaggaactgcctgtactcccgactgaccatctag

SEQ ID NO: 2 - DNA, 747 bp, Homo sapiens
atgtggctgtgccctctggccctcaccctcatcttgatggcagcctctggtgctgcgtgcgaagtgaaggacgtttgtgttggaagccctggtatccccggcactcctggatcccacggcctgccaggcagggacgggagagatggtgtcaaaggagaccctggccctccaggccccatgggtccgcctggagaaacaccatgtcctcctgggaataatgggctgcctggagcccctggtgtccctggagagcgtggagagaagggggaggctggcgagagaggccctccagggcttccagctcatctagatgaggagctccaagccacactccacgacttcagacatcaaatcctgcagacaaggggagccctcagtctgcagggctccataatgacagtaggagagaaggtcttctccagcaatgggcagtccatcacttttgatgccattcaggaggcatgtgccagagcaggcggccgcattgctgtcccaaggaatccagaggaaaatgaggccattgcaagcttcgtgaagaagtacaacacatatgcctatgtaggcctgactgagggtcccagccctggagacttccgctactcagatgggacccctgtaaactacaccaactggtaccgaggggagcctgcaggtcggggaaaagagcagtgtgtggagatgtacacagatgggcagtggaatgacaggaactgcctgtactcccgactgaccatctgtgagttctga

SEQ ID NO: 3 - protein, 248 aa, Homo sapiens
MWLCPLALNLILMAASGAVCEVKDVCVGSPGIPGTPGSHGLPGRDGRDGLKGDPGPPGPMGPPGEMPCPPGNDGLPGAPGIPGECGEKGEPGERGPPGLPAHLDEELQATLHDFRHQILQTRGALSLQGSIMTVGEKVFSSNGQSITFDAIQEACARAGGRIAVPRNPEENEAIASFVKKYNTYAYVGLTEGPSPGDFRYSDGTPVNYTNWYRGEPAGRGKEQCVEMYTDGQWNDRNCLYSRLTICEF

SEQ ID NO: 4 - protein, 248 aa, Homo sapiens [sequence omitted]

SEQ ID NO: 5 - DNA, 1128 bp, Homo sapiens
atgctgctcttcctcctctctgcactggtcctgctcacacagcccctgggctacctggaagcagaaatgaagacctactcccacagaacaatgcccagtgcttgcaccctggtcatgtgtagctcagtggagagtggcctgcctggtcgcgatggacgggatgggagagagggccctcggggcgagaagggggacccaggtttgccaggagctgcagggcaagcagggatgcctggacaagctggcccagttgggcccaaaggggacaatggctctgttggagaacctggaccaaagggagacactgggccaagtggacctccaggacctcccggtgtgcctggtccagctggaagagaaggtcccctggggaagcaggggaacataggacctcagggcaagccaggcccaaaaggagaagctgggcccaaaggagaagtaggtgccccaggcatgcagggctcggcaggggcaagaggcctcgcaggccctaagggagagcgaggtgtccctggtgagcgtggagtccctggaaacacaggggcagcagggtctgctggagccatgggtccccagggaagtccaggtgccaggggacccccgggattgaagggggacaaaggcattcctggagacaaaggagcaaagggagaaagtgggcttccagatgttgcttctctgaggcagcaggttgaggccttacagggacaagtacagcacctccaggctgctttctctcagtataagaaagttgagctcttcccaaatggccaaagtgtcggggagaagattttcaagacagcaggctttgtaaaaccatttacggaggcacagctgctgtgcacacaggctggtggacagttggcctctccacgctctgccgctgagaatgccgccttgcaacagctggtcgtagctaagaacgaggctgctttcctgagcatgactgattccaagacagagggcaagttcacctaccccacaggagagtccctggtctattccaactgggccccaggggagcccaacgatgatggcgggtcagaggactgtgtggagatcttcaccaatggcaagtggaatgacagggcttgtggagaaaagcgtcttgtggtctgcgagttctga

SEQ ID NO: 6 - protein, 375 aa, Homo sapiens
MLLFLLSALVLLTQPLGYLEAEMKTYSHRTMPSACTLVMCSSVESGLPGRDGRDGREGPRGEKGDPGLPGAAGQAGMPGQAGPVGPKGDNGSVGEPGPKGDTGPSGPPGPPGVPGPAGREGALGKQGNIGPQGKPGPKGEAGPKGEVGAPGMQGSAGARGLAGPKGERGVPGERGVPGNTGAAGSAGAMGPQGSPGARGPPGLKGDKGIPGDKGAKGESGLPDVASLRQQVEALQGQVQHLQAAFSQYKKVELFPNGQSVGEKIFKTAGFVKPFTEAQLLCTQAGGQLASPRSAAENAALQQLVVAKNEAAFLSMTDSKTEGKFTYPTGESLVYSNWAPGEPNDDGGSEDCVEIFTNGKWNDRACGEKRLVVCEF

SEQ ID NO: 7 - DNA, 25 bp, Artificial Sequence
  Description of Artificial Sequence Synthetic primer
gcagagatgggagagatggtatcaa

SEQ ID NO: 8 - DNA, 24 bp, Artificial Sequence
  Description of Artificial Sequence Synthetic primer
atggacctccattagcatgtggga

SEQ ID NO: 9 - DNA, 20 bp, Artificial Sequence
  Description of Artificial Sequence Synthetic primer
tgaatgaactgcaggacgag

SEQ ID NO: 10 - DNA, 20 bp, Artificial Sequence
  Description of Artificial Sequence Synthetic primer
atactttctcggcaggagca

SEQ ID NO: 11 - DNA, 747 bp, Bos sp.
atgctgctgtgctctttgacccttaccctcctctggatggtggcttctggcctcgagtgcgatgtcaaggaagtttgtcttggaagccctggcattcctggcactcctggatcccatggcctgccaggaagagatgggagagatggtatcaaaggagaccctgggcctccaggccccatgggcccccctggaggaatgccaggcctccctgggcgtgatgggatgactggagcccctggcctccctggagagcgtggagaaaagggagagcctggcgagagaggtcctccagggtttccagcatatctagatgaagagctccagggcacactccatgagatcagacatcaagtcctgcagtcacagggcgtcctccgtttgcaggggtccgtgctggcggtgggagagaaggtcttctctaccaatgggcagtcagtcaattttgatgccattaaagagttatgtgccagagtaggtggacatattgctgccccgaggagtccagaggagaatgaagccattgtgagcatcgtgaagaagtacaacacttatgcttacctgggcctggtcgaaggccccaccgctggagacttctattacctggatggagcccctgtgaattataccaattggtacccaggggagcccaggggccggggtaaagagaagtgtgtagaaatatacacagatggtcagtggaatgacaagaactgcctgcagtaccgactggccatctgtgagttctga

SEQ ID NO: 12 - DNA, 738 bp, Bos sp.; identical to SEQ ID NO: 1